SEQUENCE LISTING (8 sequences)

SEQ ID NO: 1 - DNA, 2603 bp, Homo sapiens
gagatcagtgcggagctgtcaaagcgagcaggggtggcgccgggagtgggaacgccacacagtgccaaatccccggctccagctcccgactcccggctcccggctcccggctcccggtgcccaatcccgggccgcagccatgaacggcgaggagcagtactacgcggccacgcagctttacaaggacccatgcgcgttccagcgaggcccggcgccggagttcagcgccagcccccctgcgtgcctgtacatgggccgccagcccccgccgccgccgccgcacccgttccctggcgccctgggcgcgctggagcagggcagccccccggacatctccccgtacgaggtgccccccctcgccgacgaccccgcggtggcgcaccttcaccaccacctcccggctcagctcgcgctcccccacccgcccgccgggcccttcccggagggagccgagccgggcgtcctggaggagcccaaccgcgtccagctgcctttcccatggatgaagtctaccaaagctcacgcgtggaaaggccagtgggcaggcggcgcctacgctgcggagccggaggagaacaagcggacgcgcacggcctacacgcgcgcacagctgctagagctggagaaggagttcctattcaacaagtacatctcacggccgcgccgggtggagctggctgtcatgttgaacttgaccgagagacacatcaagatctggttccaaaaccgccgcatgaagtggaaaaaggaggaggacaagaagcgcggcggcgggacagctgtcgggggtggcggggtcgcggagcctgagcaggactgcgccgtgacctccggcgaggagcttctggcgctgccgccgccgccgccccccggaggtgctgtgccgcccgctgcccccgttgccgcccgagagggccgcctgccgcctggccttagcgcgtcgccacagccctccagcgtcgcgcctcggcggccgcaggaaccacgatgagaggcaggagctgctcctggctgaggggcttcaaccactcgccgaggaggagcagagggcctaggaggaccccgggcgtggaccacccgccctggcagttgaatggggcggcaattgcggggcccaccttagaccgaaggggaaaacccgctctctcaggcgcatgtgccagttggggccccgcgggtagatgccggcaggccttccggaagaaaaagagccattggtttttgtagtattggggccctcttttagtgatactggattggcgttgtttgtggctgttgcgcacatccctgccctcctacagcactccaccttgggacctgtttagagaagccggctcttcaaagacaatggaaactgtaccatacacattggaaggctccctaacacacacagcggggaagctgggccgagtaccttaatctgccataaagccattcttactcgggcgacccctttaagtttagaaataattgaaaggaaatgtttgagttttcaaagatcccgtgaaattgatgccagtggaatacagtgagtcctcctcttcctcctcctcctcttccccctccccttcctcctcctcctcttcttttccctcctcttcctcttcctcctgctctcctttcctccccctcctcttttccctcctcttcctcttcctcctgctctcctttcctccccctcctctttctcctcctcctcctcttcttccccctcctctccctcctcctcttcttccccctcctctccctcctcctcttcttctccctcctcttcctcttcctcctcttccacgtgctctcctttcctccccctcctcttgctccccttcttccccgtcctcttcctcctcctcctcttcttctccctcctcttcctcctcctctttcttcctgacctctttctttctcctcctcctccttctacctccccttctcatccctcctcttcctcttctctagctgcacacttcactactgcacatcttataacttgcacccctttcttctgaggaagagaacatcttgcaaggcagggcgagcagcggcagggctggcttaggagcagtgcaagagtccctgtgctccagttccacactgctggcagggaaggcaaggggggacgggcctggatctgggggtgagggagaaagatggacccctgggtgaccactaaaccaaagatattcggaactttctatttaggatgtggacgtaattcctgttccgaggtagaggctgtgctgaagacaagcacagtggcctggtgcgccttggaaaccaacaactattcacgagccagtatgaccttcacatctttagaaattatgaaaacgtatgtgattggagggtttggaaaaccagttatcttatttaacattttaaaaattacctaacagttatttacaaacaggtctgtgcatcccaggtctgtcttcttttcaaggtctgggccttgtgctcgggttatgtttgtgggaaatgcttaataaatactgataatatgggaagagatgaaaactgattctcctcactttgtttcaaacctttctggcagtgggatgattcgaattcacttttaaaattaaattagcgtgttttgtttt

SEQ ID NO: 2 - protein, 283 aa, Homo sapiens
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 3 - DNA, 2669 bp, Homo sapiens
agccgtgggaggcggggccggccggcggcgcgggtggggcgcgggagcggtcccggagcagcccgaggcggcggccgcggggaggaggcggcgacgcgggcccggggtcgcccgagacacctggccagcggtgcccctagcgcgccgccccggagttgaccacgtgaaacttttccctgcgcccctcggcgccgccgccccgcgccggcgcccccccgcccccgccgggaccgccgcccgcggggagcaggggggggagaggcctgcagctccccccccactcccacgccgcccgtcggggcgcggccgggcgcgggccccgggcgatggccgcggagctggcgatgggcgccgagctgcccagcagcccgctggccatcgagtacgtcaacgacttcgacctgatgaagttcgaggtgaagaaggagcctcccgaggccgagcgcttctgccaccgcctgccgccaggctcgctgtcctcgacgccgctcagcacgccctgctcctccgtgccctcctcgcccagcttctgcgcgcccagcccgggcaccggcggcggcggcggcgcggggggcggcggcggctcgtctcaggccgggggcgcccccgggccgccgagcgggggccccggcgccgtcgggggcacctcggggaagccggcgctggaggatctgtactggatgagcggctaccagcatcacctcaaccccgaggcgctcaacctgacgcccgaggacgcggtggaggcgctcatcggcagcggccaccacggcgcgcaccacggcgcgcaccacccggcggccgccgcagcctacgaggctttccgcggcccgggcttcgcgggcggcggcggagcggacgacatgggcgccggccaccaccacggcgcgcaccacgccgcccaccatcaccacgccgcccaccaccaccaccaccaccaccaccaccatggcggcgcgggacacggcggtggcgcgggccaccacgtgcgcctggaggagcgcttctccgacgaccagctggtgtccatgtcggtgcgcgagctgaaccggcagctccgcggcttcagcaaggaggaggtcatccggctcaagcagaagcggcgcacgctcaagaaccgcggctacgcgcagtcctgccgcttcaagcgggtgcagcagcggcacattctggagagcgagaagtgccaactccagagccaggtggagcagctgaagctggaggtggggcgcctggccaaagagcgggacctgtacaaggagaaatacgagaagctggcgggccggggcggccccgggagcgcgggcggggccggtttcccgcgggagccttcgccgccgcaggccggtcccggcggggccaagggcacggccgacttcttcctgtaggcgccggaccccgagcccgcgccgccgtcgccggggacaagttcgcgcaggcctctcggggcctcggctcggactccgcggtacaggacgtggacaccaggcccggcccggccgtgctggccccggtgccaagtctgcgggcgcggggctggaggccccttcgctcccggtccccgttcgcgcgcgtcggcccgggtcgccgtcctgaggttgagcggagaacggtgatttctaaggaaacttgagccaggtctaacttctttccaagcgtccgcttgtacatacgttgaacgtggttctccgttcccaccttcgccctgccagcctagagggaccgcgctgccgtcccttcccgggtggcccctgcctgcccccgccctccttcgttctcttctcagcctccctttccttgccttttttaacttcccctccccgttttaaaatcggtcttattttcgaagtatttataattattatgcttggtgattagaaaagaaaaccttggaggaagccccttctttccccagccggggtccgccctcagtcgcgagtcacagcatgagtcgctcgccaggaggggcccggcccctgcctgccccctccccgcttgcccccgaccctgctaccggcgttccttggaggtcgaagccagggacgtcacccgtgctgtgtccaggcctgctgtcctactatgctcaaccgggggtggggggaggggggtgagtcctgtgctcagtcgggtgggggctggcccggatcccgagctgctgtctctctatgcaccagaacatatctgtaactcctggggaaatacatcttgttttaaccttcaagagaagtgaaagaaaaaagtaatgcacagtatttctagcagaaaatttttttttttaagaggaggcttgggccagagccttctggcatggggcgggtggagaaagtgtttttattttaatttaaattgtgtttcgttttgtttgtggaatctttctttaatgcttcgtcgctctttggactagccgggagagagggcgaggaggcgggtgctccaggccctgtaggctgggccaggcgcctgggggatctgcccgttttcggaggccctcaggggccatcagtgggattccagccgctccacacccctcccctgagcactcggagtggaaggcgcgccgactcgttgaaagttttgttgtgtagttggttttcgttgagttcttttttcatttgctacgaaactgagaaaaagaaaaaaatacacaaaataaatctgttcagatccaa

SEQ ID NO: 4 - protein, 353 aa, Homo sapiens
MAAELAMGAELPSSPLAIEYVNDFDLMKFEVKKEPPEAERFCHRLPPGSLSSTPLSTPCSSVPSSPSFCAPSPGTGGGGGAGGGGGSSQAGGAPGPPSGGPGAVGGTSGKPALEDLYWMSGYQHHLNPEALNLTPEDAVEALIGSGHHGAHHGAHHPAAAAAYEAFRGPGFAGGGGADDMGAGHHHGAHHAAHHHHAAHHHHHHHHHHGGAGHGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGSAGGAGFPREPSPPQAGPGGAKGTADFFL

SEQ ID NO: 5 - DNA, 1490 bp, Homo sapiens
gatggcccagtcctgaactccccgccatggccggcgcccccggcctgctgcgccttgcgctgctgctgctcgggatggtgggcagggccggcccccgcccccagggtgccactgtgtccctctgggagacggtgcagaaatggcgagaataccgacgccagtgccagcgctccctgactgaggatccacctcctgccacagacttgttctgcaaccggaccttcgatgaatacgcctgctggccagatggggagccaggctcgttcgtgaatgtcagctgcccctggtacctgccctgggccagcagtgtgccgcagggccacgtgtaccggttctgcacagctgaaggcctctggctgcagaaggacaactccagcctgccctggagggacttgtcggagtgcgaggagtccaagcgaggggagagaagctccccggaggagcagctcctgttcctctacatcatctacacggtgggctacgcactctccttctctgctctggttatcgcctctgcgatcctcctcggcttcagacacctgcactgcaccaggaactacatccacctgaacctgtttgcatccttcatcctgcgagcattgtccgtcttcatcaaggacgcagccctgaagtggatgtatagcacagccgcccagcagcaccagtgggatgggctcctctcctaccaggactctctgagctgccgcctggtgtttctgctcatgcagtactgtgtggcggccaattactactggctcttggtggagggcgtgtacctgtacacactgctggccttctcggtcttatctgagcaatggatcttcaggctctacgtgagcataggctggggtgttcccctgctgtttgttgtcccctggggcattgtcaagtacctctatgaggacgagggctgctggaccaggaactccaacatgaactactggctcattatccggctgcccattctctttgccattggggtgaacttcctcatctttgttcgggtcatctgcatcgtggtatccaaactgaaggccaatctcatgtgcaagacagacatcaaatgcagacttgccaagtccacgctgacactcatccccctgctggggactcatgaggtcatctttgcctttgtgatggacgagcacgcccgggggaccctgcgcttcatcaagctgtttacagagctctccttcacctccttccaggggctgatggtggccatattatactgctttgtcaacaatgaggtccagctggaatttcggaagagctgggagcgctggcggcttgagcacttgcacatccagagggacagcagcatgaagcccctcaagtgtcccaccagcagcctgagcagtggagccacggcgggcagcagcatgtacacagccacttgccaggcctcctgcagctgagactccagcgcctgccctccctggggtccttgctgcaggccgggtggccaatccaggtgggagagacactcc

SEQ ID NO: 6 - protein, 463 aa, Homo sapiens [sequence omitted]

SEQ ID NO: 7 - DNA, 1347 bp, Homo sapiens
acagatgaggttgaggttggcccacggccaggtgagaggcttccaaggcaggatacttgtgtctcagatgcggtcgcttctttcatacagcaattgccgccttgctgaggatcaaggaacctcagtgtcagatcacgccctccccccaaacttagaaattcagatggggcgcagaaatttctcttgttctgcgtgatctgcatagatggtccaagaggtggtttttccaggagcccagcacccctcctccctccgactcagacccaggagtctggccctccattgaaaggaccccaggttacatcatccattcaggctgcccttgccacgatggaattctgtagctcctgccaaatgggtcaaatatcatggttcaggcgcagggagggtgattgggcgggcctgtctgggtataaattctggagcttctgcatctatcccaaaaaacaagggtgttctgtcagctgaggatccagccgaaagaggagccaggcactcaggccacctgagtctactcacctggacaactggaatctggcaccaattctaaaccactcagcttctccgagctcacaccccggagatcacctgaggacccgagccattgatggactcggacgagaccgggttcgagcactcaggactgtgggtttctgtgctggctggtcttctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcctgaagccagaggctgtttactatgacatctcctctttatttattaggttatttatcttatttatttttttatttttcttacttgagataataaagagttccagaggaggataa

SEQ ID NO: 8 - protein, 208 aa, Homo sapiens
MDSDETGFEHSGLWVSVLAGLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

(19) United States
(12) Patent Application Publication
(10) Pub. No.: US 2023/0218780 A1
(43) Pub. Date: Jul. 13, 2023

(54) COMPOSITIONS AND METHODS FOR REPROGRAMMING SKIN TISSUE TO HAVE INSULINOGENIC AND DELIVERY FUNCTIONS

(71) Applicant: THE TRUSTEES OF INDIANA UNIVERSITY, Bloomington, IN (US)

(72) Inventors: Chandan K. SEN, Indianapolis, IN (US); Sashwati ROY, Indianapolis, IN (US); Manishekhar KUMAR, Indianapolis, IN (US); Kanhaiya SINGH, Indianapolis, IN (US)

(21) Appl. No.: 18/009,805

(22) PCT Filed: Jun. 25, 2021
(86) PCT No.: PCT/US21/39083

§ 371 Date: Dec. 12, 2022

Related U.S. Application Data
(60) Provisional application No. 63/045,440, filed on Jun. 29, 2020.

(51) Int. Cl. A61K 48/00; C12N 15/85; A61P 3/10; A61M 37/00

(52) U.S. Cl. CPC A61K 48/0016; C12N 15/85; A61P 3/10; A61M 37/0015; C12N 2800/107

(57) ABSTRACT

Disclosed herein are compositions and in vitro and in vivo methods for reprogramming post-natal (adult and juvenile) tissue into insulinogenic cells. These compositions and methods are useful for a variety of purposes, including the development of diabetes therapies.

### CROSS REFERENCE TO RELATED APPLICATIONS

This application claims priority to U.S. Provisional Patent Application No. 63/045,440 filed on Jun. 29, 2020, the disclosure of which is expressly incorporated herein.

### INCORPORATION BY REFERENCES OF MATERIAL SUBMITTED ELECTRONICALLY

Incorporated by reference in its entirety is a computer-readable nucleotide/amino acid sequence listing submitted concurrently herewith and identified as follows: 23 kilobytes ACII (Text) file named “337064_5 T25.txt,” created on Jun. 9, 2021.

### BACKGROUND

Diabetes mellitus currently afflicts at least 200 million people worldwide. Type 1 diabetes accounts for about 10% of this number, and results from autoimmune destruction of insulin-secreting β-cells in the pancreatic islets of Langerhans. Survival depends on multiple daily insulin injections. Type 2 diabetes accounts for the remaining 90% of individuals affected, and the rate of prevalence is increasing. Type 2 diabetes is often, but not always, associated with obesity, and although previously termed late-onset or adult diabetes, is now increasingly manifest in younger individuals. It is caused by a combination of insulin resistance and inadequate insulin secretion.

Diabetes, specifically Type 2 diabetes, has emerged in the twenty-first century as an epidemic of global proportions. Numerous long-term complications, including those affecting the kidneys, legs, feet, eyes, heart, nerves, and blood circulation, result from uncontrolled diabetes. Prevention of these conditions requires comprehensive treatment, requiring life style modification and medication. A number of effective anti-diabetic drugs are available and are generally safe and well tolerated. However, all currently available medications become less effective as the disease progresses, and most patients eventually require insulin.

The development of diabetes is associated with substantial losses in pancreatic islet mass. At the time of diagnosis, over 90% of islet mass has been lost in Type 1 diabetes (T1D) patients, and approximately 50% has been lost in Type 2 diabetes (T2D) patients. Many attempts have been made in quest of a potential stimulus for islet neogenesis, which is considered as the optimal treatment for both T1D and T2D. As disclosed herein compositions and methods are provided for converting a patient's own skin tissue into cells that are insulinogenic and produce insulin. Such composition and methods are believe to offer an alternative or supplemental method of treating diabetes relative to existing treatments.

### SUMMARY

In accordance with the present disclosure, compositions and in vivo methods for reprogramming somatic cells of post-natal (adult and juvenile) tissues, including for example, non-pancreatic somatic cells such as skin cells, to be insulinogenic and release insulin into a patient's blood stream are provided. In one embodiment post-natal skin tissue is reprogrammed in vivo to become insulinogenic and optionally exhibit characteristics of a pancreatic β-cell (i.e., a pancreatic β-like cell), including the production of insulin and C-peptide. More particularly, somatic cells can be transfected with a cocktail of β-cell associated peptides, or nucleic acid sequences encoding the unique cocktail of β-cell associated peptides, to induce the transfected somatic tissue (e.g., skin tissue) to be insulinogenic and produce insulin and/or insulin C-peptide in cells that otherwise do not produce insulin and/or C-peptide.

In accordance with one embodiment post-natal skin tissue is reprogrammed to be insulinogenic by transfecting cells of post-natal mammalian skin tissues with nucleic acid sequences that initiate or enhance the expression of Pancreatic And Duodenal Homeobox 1 (PDX-1), transcription factor MafA, glucagon-like peptide 1 receptor (GLP-1R) and optionally Fibroblast growth factor 21 (FGF21) within the transfected cells. In one embodiment post-natal skin tissue is transfected with a first nucleic acid sequence encoding for PDX-1, a second nucleic acid sequence encoding for transcription factor MafA, a third nucleic acid sequence encoding for GLP-1R; and optionally a fourth nucleic acid sequence comprising nucleic acid sequence encoding for FGF21, wherein each of said first, second, third and optional fourth nucleic acid sequences are operably linked to regulatory sequences that allow for expression (transcription and translation) of the proteins PDX-1, MafA, GLP-1R, and optionally FGF21 in the transfected cells. In accordance with one embodiment post-natal skin tissue is transfected with a composition comprising the first, second, third and fourth nucleic acid sequences, optionally wherein each of said first, second, third and fourth nucleic acid sequences are provided on separate plasmids. In one embodiment post-natal skin tissue is transfected with a composition comprising the first, second, third and fourth nucleic acid sequences wherein two or more of the first second, third and fourth nucleic acids are located on a single plasmid, and in one embodiment all four of the first, second, third and fourth nucleic acid sequences are located on a single plasmid.

In accordance with one embodiment a reprogramming cocktail is provided comprising a first nucleic acid sequence that comprises a sequence encoding a peptide having at least 80%, 85%, 95% or 99% sequence identity to SEQ ID NO: 2, a second nucleic acid sequence that comprises a sequence encoding a peptide having at least 80%, 85%, 95% or 99% sequence identity to SEQ ID NO: 4, a third nucleic acid sequence that comprises a sequence encoding a peptide having at least 80%, 85%, 95% or 99% sequence identity to SEQ ID NO: 6; and optionally a fourth nucleic acid sequence that comprises a sequence encoding a peptide having at least 80%, 85%, 95% or 99% sequence identity to SEQ ID NO: 8, wherein each of the first, second, third and fourth nucleic acid sequences are operably linked to regulatory sequences that allow for expression (transcription and translation) of the respective proteins PDX-1, MafA, GLP-1R, and FGF21 when the nucleic acid sequences are transfected into mammalian cells. In one embodiment the first, second, third and fourth nucleic acid sequences are operably linked to a heterologous promoter that is operable in a mammalian cell but is different from the native promoter that is operably linked to the human gene encoding the PDX-1, MafA, GLP-1R, and optionally FGF21 protein.

In one embodiment a composition for reprogramming skin tissue to be insulinogenic, and release insulin from the interior of the cells of somatic tissue (e.g., skin tissue) to the exterior of the cells is provided. In one embodiment the reprogramming composition comprises a first nucleic acid sequence comprising a sequence having at least 80%, 85%, 95% or 99% sequence identity to SEQ ID NO: 1, a second nucleic acid sequence comprising a sequence having at least 80%, 85%, 95% or 99% sequence identity to SEQ ID NO: 3, a third nucleic acid sequence comprising a sequence having at least 80%, 85%, 95% or 99% sequence identity to SEQ ID NO: 5; and a fourth nucleic acid sequence comprising a sequence encoding a peptide having at least 80%, 85%, 95% or 99% sequence identity to SEQ ID NO: 7. In one embodiment a non-viral vector is provided that comprises each of said first, second, third and fourth nucleic acid sequences wherein each of said first, second, third and fourth nucleic acid sequences are operably linked to regulatory sequences that allow for expression the encoded proteins in a mammalian cell. In one embodiment the non-viral vector comprises a single eukaryotic promoter operably linked to a multiple coding sequence that comprises said two or more of the first, second, third and fourth nucleic acid sequences wherein said multiple coding sequence further comprises internal ribosome entry sites present before each of said first, second, third and fourth nucleic acid sequences. In one embodiment the eukaryotic promoter is a heterologous promoter.

In accordance with the present disclosure the target post-natal skin tissue can be transfected with any of the reprogramming cocktails disclosed herein using any transformation technique known to those skilled in the art. In accordance with one embodiment nucleic acids of the reprogramming cocktail are introduced into the cytosol of skin cells in vivo, via nanotransfection (TNT), more particularly using the TNT device described in Example 1 and shown in FIGS. 2A-2D.

In accordance with one embodiment a kit is provided for conducting in vivo transfection of somatic tissue and inducing the cells of the somatic tissue to become insulinogenic and release insulin into the circulatory system of the patient. In one embodiment the transfected cells exhibit characteristics of a pancreatic β-cell including the production and release of insulin. In one embodiment the kit comprises a disposable nanotransfection device and a reprogramming cocktail. In one embodiment the nanotransfection device comprises a hollow microneedle array with one or more compartments for receiving a reprogramming cocktail solution or a cartridge comprising the reprogramming cocktail. In one embodiment the hollow microneedle array comprises an electrode (i.e., cathode, optionally gold-coated or silver-coated) that is positioned for contact with a solution loaded into the compartment of the device and a needle counter-electrode (i.e., anode) positioned for insertion intradermally into a patient's skin. In one embodiment the reprogramming cocktail solution comprises a first nucleic acid sequence encoding for Pancreatic And Duodenal Homeobox 1 (PDX-1), a second nucleic acid sequence encoding for transcription factor MafA, a third nucleic acid sequence encoding for glucagon-like peptide 1 receptor (GLP-1R); and optionally a fourth nucleic acid sequence comprising nucleic acid sequence encoding for Fibroblast growth factor 21 (FGF21). In accordance with one embodiment the nanotransfection device is preloaded with the reprogramming cocktail solution.

In accordance with one embodiment a method for treating Type 1 or Type2 diabetes is provided wherein a reprogramming cocktail solution comprising a first nucleic acid sequence encoding for Pancreatic And Duodenal Homeobox 1 (PDX-1), a second nucleic acid sequence encoding for transcription factor MafA, a third nucleic acid sequence encoding for glucagon-like peptide 1 receptor (GLP-1R); and optionally a fourth nucleic acid sequence comprising nucleic acid sequence encoding for Fibroblast growth factor 21 (FGF21) is introduced into the cytosol of somatic cells in vivo, optionally via nanotransfection (TNT). In one embodiment the method of treating diabetes and/or controlling blood glucose levels in a patient in need of treatment comprises the step of transfecting in vivo a reprogramming cocktail of the present disclosure into the cells of the skin tissue of a patient once a month, every 8-12 weeks, every 10 to 15 weeks or every 15 to 18 weeks.

In one embodiment a method of normalizing blood glucose levels in a subject with diabetes is provided wherein the method comprises the step of reprogramming targeted skin cells in vivo to produce insulin, wherein the method comprises contacting said target skin cells with a reprogramming composition under conditions that enhance cellular uptake of the reprogramming composition components. In one embodiment, the transfection composition comprises a first nucleic acid sequence encoding a peptide having at least 95% sequence identity to SEQ ID NO: 2, a second nucleic acid sequence encoding a peptide having at least 95% sequence identity to SEQ ID NO: 4, a third nucleic acid sequence encoding a peptide having at least 95% sequence identity to SEQ ID NO: 6; and optionally a fourth nucleic acid sequence encoding a peptide having at least 95% sequence identity to SEQ ID NO: 8, wherein the first, second, third and further nucleic acid sequences are operably linked to regulatory sequences that allow for the expression of the encoded proteins upon introduction into human skin cells. In one embodiment the cellular uptake of the nucleic acid sequences is induced through the use of nanotransfection (TNT).

### BRIEF DESCRIPTION OF THE DRAWINGS

FIG. 1 provides a schematic view of a TNT process based on a chip with a hollow microneedle array, conducted on the exfoliated skin mediated by the hollow microneedles. A plasmid DNA solution (5) is retained in a reservoir (1) and is in fluid communication with a plurality of microneedles (2) of the hollow microneedle array. The plasmid DNA solution (5) is delivered to the skin tissue, comprising the epidermis (3) and dermis (4) layers, under a square electric pulse applied at microsecond level.

FIGS. 2A-2D provide schematics of the TNT chips with various nanochannels and microneedle arrays. FIG. 2A demonstrates a TNT chip lacking any needle structures. FIG. 2B demonstrates a Type I hollow microneedle array with flat tip. FIG. 2C demonstrates a Type II hollow microneedle array with sharp tip and centered bore.

FIG. 2D demonstrates a Type III hollow microneedle array with sharp tip and off-centered bore. Cross-sectional views are also shown for each type of TNT chip.

FIGS. 3A & 3B are graphs of two separate experiments demonstrating the efficacy of the transfection cocktail comprising nucleic acid sequences encoding for Pancreatic And Duodenal Homeobox 1 (PDX-1), transcription factor MafA, glucagon-like peptide 1 receptor (GLP-1R); and Fibroblast growth factor 21 (FGF21) (the “PMGF” cocktail) in lowering blood glucose levels in streptozotocin (STZ) induced diabetic mice. Skin cell uptake of the PMGF cocktail was induced through the use of Lentiviral particles. Administration of streptozotocin (STZ) and Lentiviral particles is indicated by arrows. Blood glucose levels were significantly reduced in streptozotocin (STZ) induced diabetic mice receiving the PMGF cocktail via a Lentiviral particle (LentiPMGF) relative to control.

FIGS. 4A-4C are graphs of three separate experiments demonstrating the efficacy of a transfection cocktail comprising nucleic acid sequence encoding for Pancreatic And Duodenal Homeobox 1 (PDX-1, transcription factor MafA, glucagon-like peptide 1 receptor (GLP-1R); and Fibroblast growth factor 21 (FGF21) (the “PMGF” cocktail) in lowering blood glucose levels in streptozotocin (STZ) induced diabetic mice. Mice were divided in two different groups 1) Control, and 2) mice administered the reprogramming factors via TNT (TNT_{PMGF}). In the reprogramming cocktail 37.5 μg of each component P/M/G/F was used. Equal amount of control plasmids were delivered to the control group. The data indicates that TNT-mediated delivery of reprogramming factor cocktail leads to tissue reprogramming resulting in formation of insulinogenic cells in post-natal skin which leads to lowering of blood glucose levels in streptozotocin-induced diabetic models in mice.

FIGS. 5A-5C are graphs showing results for intraperitoneal glucose tolerance test (IPGTT). IPGTT is used to test the clearance of an intraperitoneally injected glucose load from the body. This test detects disturbances in glucose metabolism and insulin secretion. For this experiment mice were fasted and the fasting blood glucose levels were determined before a solution of glucose (D-glucose, 2 g/kg of body weight) was administered by intra-peritoneal (IP) injection. Subsequently, the blood glucose level was measured from tail vein at different time points (0, 15, 30, 60, 90 and 120 minutes) during the following 120 minutes. Intraperitoneal injection of glucose at 2 g/kg of body weight induced a rise of blood glucose concentration that returned to basal level within 120 min in TNT_{PMGF} group but not in control group. Note this experiment was conducted on STZ induced diabetic animals which were followed for 7 weeks after TNT intervention (FIGS. 4A-C). Control group is the STZ induced diabetic group treated with Sham (control) plasmid via TNT in which no lowering of glucose was seen (FIGS. 4A-C). Hence, in the control group of FIG. 5A, no blood glucose lowering effect was noticed and the mice showed elevated blood glucose in hyperglycemic range. Therefore, the baseline glucose levels for these mice were in the range of ˜550 mg/dl compared to TNT-PMGF group (˜300 mg/dl). Baseline blood glucose levels were much lower in High Responders shown in FIGS. 5B & 5C (˜200-250 mg/dl)

### DETAILED DESCRIPTION

### Definitions

In describing and claiming the invention, the following terminology will be used in accordance with the definitions set forth below.

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition. The term “purified polypeptide” is used herein to describe a polypeptide which has been separated from other compounds including, but not limited to nucleic acid molecules, lipids and carbohydrates.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

Tissue nanotransfection (TNT) is an electroporation-based technique capable of delivering nucleic acid sequences and proteins into the cytosol of cells at nanoscale. More particularly, TNT uses a highly intense and focused electric field through arrayed nanochannels, which benignly nanoporates the juxtaposing tissue cell members, and electrophoretically drives cargo (e.g., nucleic acids or proteins) into the cells.

As used herein “control elements” or “regulatory sequences” are non-translated regions of a functional gene, including enhancers, promoters, 5′ and 3′ untranslated regions, which interact with host cellular proteins to carry out transcription and translation. Such elements may vary in their strength and specificity. “Eukaryotic regulatory sequences” are non-translated regions of a functional gene, including enhancers, promoters, 5′ and 3′ untranslated regions, which interact with host cellular proteins of a eukaryotic cell to carry out transcription and translation in a eukaryotic cell including mammalian cells.

As used herein a “promoter” is a sequence or sequences of DNA that function when in a relatively fixed location in regard to the transcription start site of a gene. A “promoter” contains core elements required for basic interaction of RNA polymerase and transcription factors and can contain upstream elements and response elements.

As used herein an “Enhancer” is a sequence of DNA that functions independent of distance from the transcription start site and can be either 5′ or 3′ to the transcription unit. Furthermore, enhancers can be within an intron as well as within the coding sequence itself. They are usually between 10 and 300 bp in length, and they function in cis Enhancers function to increase transcription from nearby promoters. Enhancers, like promoters, also often contain response elements that mediate the regulation of transcription. Enhancers often determine the regulation of expression.

An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter. As used herein an exogenous sequence in reference to a cell is a sequence that has been introduced into the cell from a source external to the cell.

As used herein the term “non-coded (non-canonical) amino acid” encompasses any amino acid that is not an L-isomer of any of the following 20 amino acids: Ala, Cys, Asp, Glu, Phe, Gly, His, Ile, Lys, Leu, Met, Asn, Pro, Gln, Arg, Ser, Thr, Val, Trp, Tyr.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

The term “stringent hybridization conditions” as used herein mean that hybridization will generally occur if there is at least 95% and preferably at least 97% sequence identity between the probe and the target sequence. Examples of stringent hybridization conditions are overnight incubation in a solution comprising 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared carrier DNA such as salmon sperm DNA, followed by washing the hybridization support in 0.1×SSC at approximately 65° C. Other hybridization and wash conditions are well known and are exemplified in Sambrook et al, Molecular Cloning: A Laboratory Manual, Second Edition, Cold Spring Harbor, N.Y. (1989), particularly chapter 11.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “phosphate buffered saline” or “PBS” refers to aqueous solution comprising sodium chloride and sodium phosphate. Different formulations of PBS are known to those skilled in the art but for purposes of this invention the phrase “standard PBS” refers to a solution having have a final concentration of 137 mM NaCl, 10 mM Phosphate, 2.7 mM KCl, and a pH of 7.2-7.4.

As used herein, the term “treating” includes alleviation of the symptoms associated with a specific disorder or condition and/or preventing or eliminating said symptoms.

As used herein an “effective” amount or a “therapeutically effective amount” of a drug refers to a nontoxic but sufficient amount of the drug to provide the desired effect. The amount that is “effective” will vary from subject to subject or even within a subject overtime, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein an amino acid “substitution” refers to the replacement of one amino acid residue by a different amino acid residue.

As used herein, the term “conservative amino acid substitution” is defined herein as exchanges within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys; Ornithine (Orn)

IV. Large, aliphatic, nonpolar residues:
Met, Leu, Ile, Val, Cys, Norleucine (Nle), homocysteine (hCys)

V. Large, aromatic residues:
Phe, Tyr, Trp, acetyl phenylalanine, napthylalanine (Nal)

As used herein the term “patient” without further designation is intended to encompass any warm blooded vertebrate domesticated animal (including for example, but not limited to livestock, horses, cats, dogs and other pets) and humans receiving therapeutic care whether or not under the supervision of a physician.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W. H. Freeman and Company, New York (1993); Posttranslational Covalent Modification of Proteins, B. C. Johnson, Ed., Academic Press, New York, pp. 1-12 (1983)).

The term “amino acid sequence” refers to a series of two or more amino acids linked together via peptide bonds wherein the order of the amino acids linkages is designated by a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

“Nucleotide” as used herein is a molecule that contains a base moiety, a sugar moiety, and a phosphate moiety. Nucleotides can be linked together through their phosphate moieties and sugar moieties creating an internucleoside linkage. The term “oligonucleotide” is sometimes used to refer to a molecule that contains two or more nucleotides linked together. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). A nucleotide analog is a nucleotide that contains some type of modification to the base, sugar, and/or phosphate moieties. Modifications to nucleotides are well known in the art and would include, for example, 5-methylcytosine (5-me-C), 5 hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but are linked together through a moiety other than a phosphate moiety. Nucleotide substitutes are able to conform to a double helix type structure when interacting with the appropriate target nucleic acid.

The term “vector” or “construct” designates a DNA molecule used as a vehicle to carry foreign genetic material into another cell, where it can be replicated and/or expressed. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “delivery vehicle” defines any moiety that promote uptake of the nucleic acid by a cell, including both viral delivery systems and non-viral delivery systems such as cationic polymers, liposomes, exosomes, and nanoparticles containing nucleic acid.

The term “operably linked to” refers to the functional relationship of a nucleic acid with another nucleic acid sequence. Promoters, enhancers, transcriptional and translational stop sites, and other signal sequences are examples of nucleic acid sequences that can operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

As used herein the abbreviation “PMGF” designates a combination of one or more plasmids comprising nucleic acid sequences that encode for the proteins PDX1, MafA, GLP1R and FGF21.

### EMBODIMENTS

As disclosed herein composition and methods are provided for transfecting tissues and cells to convert a non-insulin producing post-natal tissue into a tissue that produces and delivers functional insulin peptides to a patient's circulatory system. The present disclosure is based on the discovery that cells modified to express a combination of proteins including PDX1, MafA, GLP1R and FGF21 will express signature beta cell markers, insulin and C-peptide. Accordingly, elevating cellular concentrations of the proteins PDX1, MafA, GLP1R and FGF21, has been found to be effective in non-invasive insulinogenic reprogramming of skin. Furthermore, the overexpression of PDX1, MafA, GLP1R and FGF21 in cells of mammalian skin reprograms skin tissue into insulin-producing tissue in vivo wherein the level of insulin production in such reprogrammed tissue can be sufficient to moderate blood glucose levels in streptozotocin-induced diabetic mice towards normalized levels.

Amino acid sequences (Table 1) and nucleic acid sequences (Table 2) encoding transcription factors PDX-1, MafA, GLP1R and FGF21 are known in the art. While human sequences are disclosed herein, other mammalian forms of these proteins, including human forms, are known in the art and can be used in the disclosed methods.

Amino acid sequences having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequences shown in Table 1 are included in the invention.

Nucleotide sequences that hybridizes to nucleic acid sequence shown in Table 2 under stringent hybridization conditions are included in the invention.

TABLE 1
Amino Acid Sequences

| Transcription Factor | Organism | Amino Acid Sequence |
|---|---|---|
| pancreas/duodenum | human | MNGEEQYYAATQLYKDPCAFQRGPAPEFSASPPACLYMGRQ |
| homeobox protein | | PPPPPPHPFPGALGALEQGSPPDISPYEVPPLADDPAVAHL |
| 1 (PDX-1) | | HHHLPAQLALPHPPAGPFPEGAEPGVLEEPNRVQLPFPWMK |
| | | STKAHAWKGQWAGGAYAAEPEENKRTRTAYTRAQLLELEKE |
| | | FLFNKYISRPRRVELAVMLNLTERHIKIWFQNRRMKWKKEE |
| | | DKKRGGGTAVGGGGVAEPEQDCAVTSGEELLALPPPPPPGG |
| | | AVPPAAPVAAREGRLPPGLSASPQPSSVAPRRPQEPR |
| | | (SEQ ID NO: 2) |
| MAFbZIP | human | MAAELAMGAELPSSPLAIEYVNDFDLMKFEVKKEPPEAERF |
| transcription factor | | CHRLPPGSLSSTPLSTPCSSVPSSPSFCAPSPGTGGGGGAG |
| A (MafA) | | GGGGSSQAGGAPGPPSGGPGAVGGTSGKPALEDLYWMSGYQ |
| | | HHLNPEALNLTPEDAVEALIGSGHHGAHHGAHHPAAAAAYE |
| | | AFRGPGFAGGGGADDMGAGHHHGAHHAAHHHHAAHHHHHHH |
| | | HHHGGAGHGGGAGHHVRLEERFSDDQLVSMSVRELNRQLRG |
| | | FSKEEVIRLKQKRRTLKNRGYAQSCRFKRVQQRHILESEKC |
| | | QLQSQVEQLKLEVGRLAKERDLYKEKYEKLAGRGGPGSAGG |
| | | AGFPREPSPPQAGPGGAKGTADFFL |
| | | (SEQ ID NO: 4) |
| Glucagon like | human | MAGAPGLLRLALLLLGMVGRAGPRPQGATVSLWETVQKWRE |
| peptide 1 receptor | | YRRQCQRSLTEDPPPATDLFCNRTFDEYACWPDGEPGSFVN |
| (GLPIR) | | VSCPWYLPWASSVPQGHVYRFCTAEGLWLQKDNSSLPWRDL |
| | | SECEESKRGERSSPEEQLLFLYIIYTVGYALSFSALVIASA |
| | | ILLGFRHLHCTRNYIHLNLFASFILRALSVFIKDAALKWMY |
| | | STAAQQHQWDGLLSYQDSLSCRLVFLLMQYCVAANYYWLLV |
| | | EGVYLYTLLAFSVLSEQWIFRLYVSIGWGVPLLFVVPWGIV |
| | | KYLYEDEGCWTRNSNMNYWLIIRLPILFAIGVNFLIFVRVI |
| | | CIVVSKLKANLMCKTDIKCRLAKSTLTLIPLLGTHEVIFAF |
| | | VMDEHARGTLRFIKLFTELSFTSFQGLMVAILYCFVNNEVQ |
| | | LEFRKSWERWRLEHLHIQRDSSMKPLKCPTSSLSSGATAGS |
| | | SMYTATCQASCS (SEQ ID NO: 6) |
| Fibroblast growth | | MDSDETGFEHSGLWVSVLAGLLGACQAHPIPDSSPLLQFGG |
| factor (FGF21) | | QVRQRYLYTDDAQQTEAHLEIREDGTVGGAADQSPESLLQL |
| | | KALKPGVIQILGVKTSRFLCQRPDGALYGSLHFDPEACSFR |
| | | ELLLEDGYNVYQSEAHGLPLHLPGNKSPHRDPAPRGPARFL |
| | | PLPGLPPALPEPPGILAPQPPDVGSSDPLSMVGPSQGRSPS |
| | | YAS (SEQ ID NO: 8) |

TABLE 2
Nucleotide Sequences

| Transcription Factor | Organism | Nucleotide Sequence |
|---|---|---|
| pancreas/duodenum | human | GAGATCAGTG CGGAGCTGTC AAAGCGAGCA |
| homeobox protein | | GGGGTGGCGC CGGGAGTGGG AACGCCACAC |
| 1 (PDX-1) | | AGTGCCAAAT CCCCGGCTCC AGCTCCCGAC |
| | | TCCCGGCTCC CGGCTCCCGG CTCCCGGTGC |
| | | CCAATCCCGG GCCGCAGCCA TGAACGGCGA |
| | | GGAGCAGTAC TACGCGGCCA CGCAGCTTTA |
| | | CAAGGACCCA TGCGCGTTCC AGCGAGGCCC |
| | | GGCGCCGGAG TTCAGCGCCA GCCCCCCTGC |
| | | GTGCCTGTAC ATGGGCCGCC AGCCCCCGCC |
| | | GCCGCCGCCG CACCCGTTCC CTGGCGCCCT |
| | | GGGCGCGCTG GAGCAGGGCA GCCCCCCGGA |
| | | CATCTCCCCG TACGAGGTGC CCCCCCTCGC |
| | | CGACGACCCC GCGGTGGCGC ACCTTCACCA |
| | | CCACCTCCCG GCTCAGCTCG CGCTCCCCCA |
| | | CCCGCCCGCC GGGCCCTTCC CGGAGGGAGC |
| | | CGAGCCGGGC GTCCTGGAGG AGCCCAACCG |
| | | CGTCCAGCTG CCTTTCCCAT GGATGAAGTC |
| | | TACCAAAGCT CACGCGTGGA AAGGCCAGTG |
| | | GGCAGGCGGC GCCTACGCTG CGGAGCCGGA |
| | | GGAGAACAAG CGGACGCGCA CGGCCTACAC |
| | | GCGCGCACAG CTGCTAGAGC TGGAGAAGGA |
| | | GTTCCTATTC AACAAGTACA TCTCACGGCC |
| | | GCGCCGGGTG GAGCTGGCTG TCATGTTGAA |
| | | CTTGACCGAG AGACACATCA AGATCTGGTT |
| | | CCAAAACCGC CGCATGAAGT GGAAAAAGGA |
| | | GGAGGACAAG AAGCGCGGCG GCGGGACAGC |
| | | TGTCGGGGGT GGCGGGGTCG CGGAGCCTGA |
| | | GCAGGACTGC GCCGTGACCT CCGGCGAGGA |
| | | GCTTCTGGCG CTGCCGCCGC CGCCGCCCCC |
| | | CGGAGGTGCT GTGCCGCCCG CTGCCCCCGT |
| | | TGCCGCCCGA GAGGGCCGCC TGCCGCCTGG |
| | | CCTTAGCGCG TCGCCACAGC CCTCCAGCGT |
| | | CGCGCCTCGG CGGCCGCAGG AACCACGATG |
| | | AGAGGCAGGA GCTGCTCCTG GCTGAGGGGC |
| | | TTCAACCACT CGCCGAGGAG GAGCAGAGGG |
| | | CCTAGGAGGA CCCCGGGCGT GGACCACCCG |
| | | CCCTGGCAGT TGAATGGGGC GGCAATTGCG |
| | | GGGCCCACCT TAGACCGAAG GGGAAAACCC |
| | | GCTCTCTCAG GCGCATGTGC CAGTTGGGGC |
| | | CCCGCGGGTA GATGCCGGCA GGCCTTCCGG |
| | | AAGAAAAAGA GCCATTGGTT TTTGTAGTAT |
| | | TGGGGCCCTC TTTTAGTGAT ACTGGATTGG |
| | | CGTTGTTTGT GGCTGTTGCG CACATCCCTG |
| | | CCCTCCTACA GCACTCCACC TTGGGACCTG |
| | | TTTAGAGAAG CCGGCTCTTC AAAGACAATG |
| | | GAAACTGTAC CATACACATT GGAAGGCTCC |
| | | CTAACACACA CAGCGGGGAA GCTGGGCCGA |
| | | GTACCTTAAT CTGCCATAAA GCCATTCTTA |
| | | CTCGGGCGAC CCCTTTAAGT TTAGAAATAA |
| | | TTGAAAGGAA ATGTTTGAGT TTTCAAAGAT |
| | | CCCGTGAAAT TGATGCCAGT GGAATACAGT |
| | | GAGTCCTCCT CTTCCTCCTC CTCCTCTTCC |
| | | CCCTCCCCTT CCTCCTCCTC CTCTTCTTTT |
| | | CCCTCCTCTT CCTCTTCCTC CTGCTCTCCT |
| | | TTCCTCCCCC TCCTCTTTTC CCTCCTCTTC |
| | | CTCTTCCTCC TGCTCTCCTT TCCTCCCCCT |
| | | CCTCTTTCTC CTCCTCCTCC TCTTCTTCCC |
| | | CCTCCTCTCC CTCCTCCTCT TCTTCCCCCT |
| | | CCTCTCCCTC CTCCTCTTCT TCTCCCTCCT |
| | | CTTCCTCTTC CTCCTCTTCC ACGTGCTCTC |
| | | CTTTCCTCCC CCTCCTCTTG CTCCCCTTCT |
| | | TCCCCGTCCT CTTCCTCCTC CTCCTCTTCT |
| | | TCTCCCTCCT CTTCCTCCTC CTCTTTCTTC |
| | | CTGACCTCTT TCTTTCTCCT CCTCCTCCTT |
| | | CTACCTCCCC TTCTCATCCC TCCTCTTCCT |
| | | CTTCTCTAGC TGCACACTTC ACTACTGCAC |
| | | ATCTTATAAC TTGCACCCCT TTCTTCTGAG |
| | | GAAGAGAACA TCTTGCAAGG CAGGGCGAGC |
| | | AGCGGCAGGG CTGGCTTAGG AGCAGTGCAA |
| | | GAGTCCCTGT GCTCCAGTTC CACACTGCTG |
| | | GCAGGGAAGG CAAGGGGGGA CGGGCCTGGA |
| | | TCTGGGGGTG AGGGAGAAAG ATGGACCCCT |
| | | GGGTGACCAC TAAACCAAAG ATATTCGGAA |
| | | CTTTCTATTT AGGATGTGGA CGTAATTCCT |
| | | GTTCCGAGGT AGAGGCTGTG CTGAAGACAA |
| | | GCACAGTGGC CTGGTGCGCC TTGGAAACCA |
| | | ACAACTATTC ACGAGCCAGT ATGACCTTCA |
| | | CATCTTTAGA AATTATGAAA ACGTATGTGA |
| | | TTGGAGGGTT TGGAAAACCA GTTATCTTAT |
| | | TTAACATTTT AAAAATTACC TAACAGTTAT |
| | | TTACAAACAG GTCTGTGCAT CCCAGGTCTG |
| | | TCTTCTTTTC AAGGTCTGGG CCTTGTGCTC |
| | | GGGTTATGTT TGTGGGAAAT GCTTAATAAA |
| | | TACTGATAAT ATGGGAAGAG ATGAAAACTG |
| | | ATTCTCCTCA CTTTGTTTCA AACCTTTCTG |
| | | GCAGTGGGAT GATTCGAATT CACTTTTAAA |
| | | ATTAAATTAG CGTGTTTTGT TTT |
| | | (SEQ ID NO: 1) |
| MAF bZIP | human | AGCCGTGGGA GGCGGGGCCG GCCGGCGGCG |
| transcription factor | | CGGGTGGGGC GCGGGAGCGG TCCCGGAGCA |
| A (MafA) | | GCCCGAGGCG GCGGCCGCGG GGAGGAGGCG |
| | | GCGACGCGGG CCCGGGGTCG CCCGAGACAC |
| | | CTGGCCAGCG GTGCCCCTAG CGCGCCGCCC |
| | | CGGAGTTGAC CACGTGAAAC TTTTCCCTGC |
| | | GCCCCTCGGC GCCGCCGCCC CGCGCCGGCG |
| | | CCCCCCCGCC CCCGCCGGGA CCGCCGCCCG |
| | | CGGGGAGCAG GGGGGGGAGA GGCCTGCAGC |
| | | TCCCCCCCCA CTCCCACGCC GCCCGTCGGG |
| | | GCGCGGCCGG GCGCGGGCCC CGGGCGATGG |
| | | CCGCGGAGCT GGCGATGGGC GCCGAGCTGC |
| | | CCAGCAGCCC GCTGGCCATC GAGTACGTCA |
| | | ACGACTTCGA CCTGATGAAG TTCGAGGTGA |
| | | AGAAGGAGCC TCCCGAGGCC GAGCGCTTCT |
| | | GCCACCGCCT GCCGCCAGGC TCGCTGTCCT |
| | | CGACGCCGCT CAGCACGCCC TGCTCCTCCG |
| | | TGCCCTCCTC GCCCAGCTTC TGCGCGCCCA |
| | | GCCCGGGCAC CGGCGGCGGC GGCGGCGCGG |
| | | GGGGCGGCGG CGGCTCGTCT CAGGCCGGGG |
| | | GCGCCCCCGG GCCGCCGAGC GGGGGCCCCG |
| | | GCGCCGTCGG GGGCACCTCG GGGAAGCCGG |
| | | CGCTGGAGGA TCTGTACTGG ATGAGCGGCT |
| | | ACCAGCATCA CCTCAACCCC GAGGCGCTCA |
| | | ACCTGACGCC CGAGGACGCG GTGGAGGCGC |
| | | TCATCGGCAG CGGCCACCAC GGCGCGCACC |
| | | ACGGCGCGCA CCACCCGGCG GCCGCCGCAG |
| | | CCTACGAGGC TTTCCGCGGC CCGGGCTTCG |
| | | CGGGCGGCGG CGGAGCGGAC GACATGGGCG |
| | | CCGGCCACCA CCACGGCGCG CACCACGCCG |
| | | CCCACCATCA CCACGCCGCC CACCACCACC |
| | | ACCACCACCA CCACCACCAT GGCGGCGCGG |
| | | GACACGGCGG TGGCGCGGGC CACCACGTGC |
| | | GCCTGGAGGA GCGCTTCTCC GACGACCAGC |
| | | TGGTGTCCAT GTCGGTGCGC GAGCTGAACC |
| | | GGCAGCTCCG CGGCTTCAGC AAGGAGGAGG |
| | | TCATCCGGCT CAAGCAGAAG CGGCGCACGC |
| | | TCAAGAACCG CGGCTACGCG CAGTCCTGCC |
| | | GCTTCAAGCG GGTGCAGCAG CGGCACATTC |
| | | TGGAGAGCGA GAAGTGCCAA CTCCAGAGCC |
| | | AGGTGGAGCA GCTGAAGCTG GAGGTGGGGC |
| | | GCCTGGCCAA AGAGCGGGAC CTGTACAAGG |
| | | AGAAATACGA GAAGCTGGCG GGCCGGGGCG |
| | | GCCCCGGGAG CGCGGGCGGG GCCGGTTTCC |
| | | CGCGGGAGCC TTCGCCGCCG CAGGCCGGTC |
| | | CCGGCGGGGC CAAGGGCACG GCCGACTTCT |
| | | TCCTGTAGGC GCCGGACCCC GAGCCCGCGC |
| | | CGCCGTCGCC GGGGACAAGT TCGCGCAGGC |
| | | CTCTCGGGGC CTCGGCTCGG ACTCCGCGGT |
| | | ACAGGACGTG GACACCAGGC CCGGCCCGGC |
| | | CGTGCTGGCC CCGGTGCCAA GTCTGCGGGC |
| | | GCGGGGCTGG AGGCCCCTTC GCTCCCGGTC |
| | | CCCGTTCGCG CGCGTCGGCC CGGGTCGCCG |
| | | TCCTGAGGTT GAGCGGAGAA CGGTGATTTC |
| | | TAAGGAAACT TGAGCCAGGT CTAACTTCTT |
| | | TCCAAGCGTC CGCTTGTACA TACGTTGAAC |
| | | GTGGTTCTCC GTTCCCACCT TCGCCCTGCC |
| | | AGCCTAGAGG GACCGCGCTG CCGTCCCTTC |
| | | CCGGGTGGCC CCTGCCTGCC CCCGCCCTCC |
| | | TTCGTTCTCT TCTCAGCCTC CCTTTCCTTG |
| | | CCTTTTTTAA CTTCCCCTCC CCGTTTTAAA |
| | | ATCGGTCTTA TTTTCGAAGT ATTTATAATT |
| | | ATTATGCTTG GTGATTAGAA AAGAAAACCT |
| | | TGGAGGAAGC CCCTTCTTTC CCCAGCCGGG |
| | | GTCCGCCCTC AGTCGCGAGT CACAGCATGA |
| | | GTCGCTCGCC AGGAGGGGCC CGGCCCCTGC |
| | | CTGCCCCCTC CCCGCTTGCC CCCGACCCTG |
| | | CTACCGGCGT TCCTTGGAGG TCGAAGCCAG |
| | | GGACGTCACC CGTGCTGTGT CCAGGCCTGC |
| | | TGTCCTACTA TGCTCAACCG GGGGTGGGGG |
| | | GAGGGGGGTG AGTCCTGTGC TCAGTCGGGT |
| | | GGGGGCTGGC CCGGATCCCG AGCTGCTGTC |
| | | TCTCTATGCA CCAGAACATA TCTGTAACTC |
| | | CTGGGGAAAT ACATCTTGTT TTAACCTTCA |
| | | AGAGAAGTGA AAGAAAAAAG TAATGCACAG |
| | | TATTTCTAGC AGAAAATTTT TTTTTTTAAG |
| | | AGGAGGCTTG GGCCAGAGCC TTCTGGCATG |
| | | GGGCGGGTGG AGAAAGTGTT TTTATTTTAA |
| | | TTTAAATTGT GTTTCGTTTT GTTTGTGGAA |
| | | TCTTTCTTTA ATGCTTCGTC GCTCTTTGGA |
| | | CTAGCCGGGA GAGAGGGCGA GGAGGCGGGT |
| | | GCTCCAGGCC CTGTAGGCTG GGCCAGGCGC |
| | | CTGGGGGATC TGCCCGTTTT CGGAGGCCCT |
| | | CAGGGGCCAT CAGTGGGATT CCAGCCGCTC |
| | | CACACCCCTC CCCTGAGCAC TCGGAGTGGA |
| | | AGGCGCGCCG ACTCGTTGAA AGTTTTGTTG |
| | | TGTAGTTGGT TTTCGTTGAG TTCTTTTTTC |
| | | ATTTGCTACG AAACTGAGAA AAAGAAAAAA |
| | | ATACACAAAA TAAATCTGTT CAGATCCAA |
| | | (SEQ ID NO: 3) |
| Glucagon like | human | GATGGCCCAG TCCTGAACTC CCCGCCATGG |
| peptide 1 receptor | | CCGGCGCCCC CGGCCTGCTG CGCCTTGCGC |
| (GLP1R) | | TGCTGCTGCT CGGGATGGTG GGCAGGGCCG |
| | | GCCCCCGCCC CCAGGGTGCC ACTGTGTCCC |
| | | TCTGGGAGAC GGTGCAGAAA TGGCGAGAAT |
| | | ACCGACGCCA GTGCCAGCGC TCCCTGACTG |
| | | AGGATCCACC TCCTGCCACA GACTTGTTCT |
| | | GCAACCGGAC CTTCGATGAA TACGCCTGCT |
| | | GGCCAGATGG GGAGCCAGGC TCGTTCGTGA |
| | | ATGTCAGCTG CCCCTGGTAC CTGCCCTGGG |
| | | CCAGCAGTGT GCCGCAGGGC CACGTGTACC |
| | | GGTTCTGCAC AGCTGAAGGC CTCTGGCTGC |
| | | AGAAGGACAA CTCCAGCCTG CCCTGGAGGG |
| | | ACTTGTCGGA GTGCGAGGAG TCCAAGCGAG |
| | | GGGAGAGAAG CTCCCCGGAG GAGCAGCTCC |
| | | TGTTCCTCTA CATCATCTAC ACGGTGGGCT |
| | | ACGCACTCTC CTTCTCTGCT CTGGTTATCG |
| | | CCTCTGCGAT CCTCCTCGGC TTCAGACACC |
| | | TGCACTGCAC CAGGAACTAC ATCCACCTGA |
| | | ACCTGTTTGC ATCCTTCATC CTGCGAGCAT |
| | | TGTCCGTCTT CATCAAGGAC GCAGCCCTGA |
| | | AGTGGATGTA TAGCACAGCC GCCCAGCAGC |
| | | ACCAGTGGGA TGGGCTCCTC TCCTACCAGG |
| | | ACTCTCTGAG CTGCCGCCTG GTGTTTCTGC |
| | | TCATGCAGTA CTGTGTGGCG GCCAATTACT |
| | | ACTGGCTCTT GGTGGAGGGC GTGTACCTGT |
| | | ACACACTGCT GGCCTTCTCG GTCTTATCTG |
| | | AGCAATGGAT CTTCAGGCTC TACGTGAGCA |
| | | TAGGCTGGGG TGTTCCCCTG CTGTTTGTTG |
| | | TCCCCTGGGG CATTGTCAAG TACCTCTATG |
| | | AGGACGAGGG CTGCTGGACC AGGAACTCCA |
| | | ACATGAACTA CTGGCTCATT ATCCGGCTGC |
| | | CCATTCTCTT TGCCATTGGG GTGAACTTCC |
| | | TCATCTTTGT TCGGGTCATC TGCATCGTGG |
| | | TATCCAAACT GAAGGCCAAT CTCATGTGCA |
| | | AGACAGACAT CAAATGCAGA CTTGCCAAGT |
| | | CCACGCTGAC ACTCATCCCC CTGCTGGGGA |
| | | CTCATGAGGT CATCTTTGCC TTTGTGATGG |
| | | ACGAGCACGC CCGGGGGACC CTGCGCTTCA |
| | | TCAAGCTGTT TACAGAGCTC TCCTTCACCT |
| | | CCTTCCAGGG GCTGATGGTG GCCATATTAT |
| | | ACTGCTTTGT CAACAATGAG GTCCAGCTGG |
| | | AATTTCGGAA GAGCTGGGAG CGCTGGCGGC |
| | | TTGAGCACTT GCACATCCAG AGGGACAGCA |
| | | GCATGAAGCC CCTCAAGTGT CCCACCAGCA |
| | | GCCTGAGCAG TGGAGCCACG GCGGGCAGCA |
| | | GCATGTACAC AGCCACTTGC CAGGCCTCCT |
| | | GCAGCTGAGA CTCCAGCGCC TGCCCTCCCT |
| | | GGGGTCCTTG CTGCAGGCCG GGTGGCCAAT |
| | | CCAGGTGGGA GAGACACTCC |
| | | (SEQ ID NO: 5) |
| Fibroblast growth | human | ACAGATGAGG TTGAGGTTGG CCCACGGCCA |
| factor (FGF21) | | GGTGAGAGGC TTCCAAGGCA GGATACTTGT |
| | | GTCTCAGATG CGGTCGCTTC TTTCATACAG |
| | | CAATTGCCGC CTTGCTGAGG ATCAAGGAAC |
| | | CTCAGTGTCA GATCACGCCC TCCCCCCAAA |
| | | CTTAGAAATT CAGATGGGGC GCAGAAATTT |
| | | CTCTTGTTCT GCGTGATCTG CATAGATGGT |
| | | CCAAGAGGTG GTTTTTCCAG GAGCCCAGCA |
| | | CCCCTCCTCC CTCCGACTCA GACCCAGGAG |
| | | TCTGGCCCTC CATTGAAAGG ACCCCAGGTT |
| | | ACATCATCCA TTCAGGCTGC CCTTGCCACG |
| | | ATGGAATTCT GTAGCTCCTG CCAAATGGGT |
| | | CAAATATCAT GGTTCAGGCG CAGGGAGGGT |
| | | GATTGGGCGG GCCTGTCTGG GTATAAATTC |
| | | TGGAGCTTCT GCATCTATCC CAAAAAACAA |
| | | GGGTGTTCTG TCAGCTGAGG ATCCAGCCGA |
| | | AAGAGGAGCC AGGCACTCAG GCCACCTGAG |
| | | TCTACTCACC TGGACAACTG GAATCTGGCA |
| | | CCAATTCTAA ACCACTCAGC TTCTCCGAGC |
| | | TCACACCCCG GAGATCACCT GAGGACCCGA |
| | | GCCATTGATG GACTCGGACG AGACCGGGTT |
| | | CGAGCACTCA GGACTGTGGG TTTCTGTGCT |
| | | GGCTGGTCTT CTGCTGGGAG CCTGCCAGGC |
| | | ACACCCCATC CCTGACTCCA GTCCTCTCCT |
| | | GCAATTCGGG GGCCAAGTCC GGCAGCGGTA |
| | | CCTCTACACA GATGATGCCC AGCAGACAGA |
| | | AGCCCACCTG GAGATCAGGG AGGATGGGAC |
| | | GGTGGGGGGC GCTGCTGACC AGAGCCCCGA |
| | | AAGTCTCCTG CAGCTGAAAG CCTTGAAGCC |
| | | GGGAGTTATT CAAATCTTGG GAGTCAAGAC |
| | | ATCCAGGTTC CTGTGCCAGC GGCCAGATGG |
| | | GGCCCTGTAT GGATCGCTCC ACTTTGACCC |
| | | TGAGGCCTGC AGCTTCCGGG AGCTGCTTCT |
| | | TGAGGACGGA TACAATGTTT ACCAGTCCGA |
| | | AGCCCACGGC CTCCCGCTGC ACCTGCCAGG |
| | | GAACAAGTCC CCACACCGGG ACCCTGCACC |
| | | CCGAGGACCA GCTCGCTTCC TGCCACTACC |
| | | AGGCCTGCCC CCCGCACTCC CGGAGCCACC |
| | | CGGAATCCTG GCCCCCCAGC CCCCCGATGT |
| | | GGGCTCCTCG GACCCTCTGA GCATGGTGGG |
| | | ACCTTCCCAG GGCCGAAGCC CCAGCTACGC |
| | | TTCCTGAAGC CAGAGGCTGT TTACTATGAC |
| | | ATCTCCTCTT TATTTATTAG GTTATTTATC |
| | | TTATTTATTT TTTTATTTTT CTTACTTGAG |
| | | ATAATAAAGA GTTCCAGAGG AGGATAA |
| | | (SEQ ID NO: 7) |

The polynucleotides may be delivered to the skin tissue via a gene gun, a microparticle or nanoparticle suitable for such delivery, a liposome or other membrane bound vesicle suitable for such delivery, injection of naked DNA or viral-based vectors, or transfection by electroporation, using a three-dimensional nanochannel electroporation, a tissue nanotransfection (TNT) device, or a deep-topical tissue nanoelectroinjection device. In some embodiments, a viral vector can be used. However, in other embodiments, the polynucleotides are not delivered virally.

The inventive compositions and methods for reprogramming post-natal somatic tissues, including non-pancreatic somatic tissue such as skin tissue, into insulinogenic cells is applicable both in vitro and in vivo.

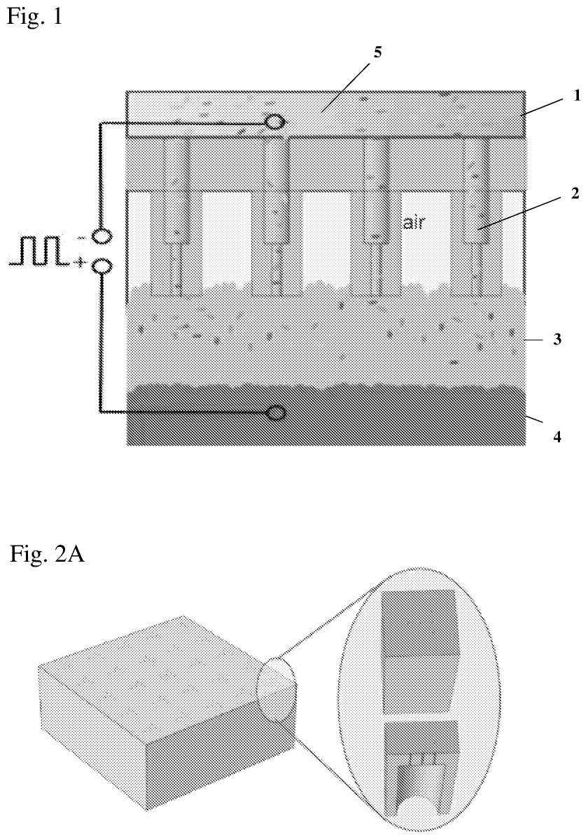

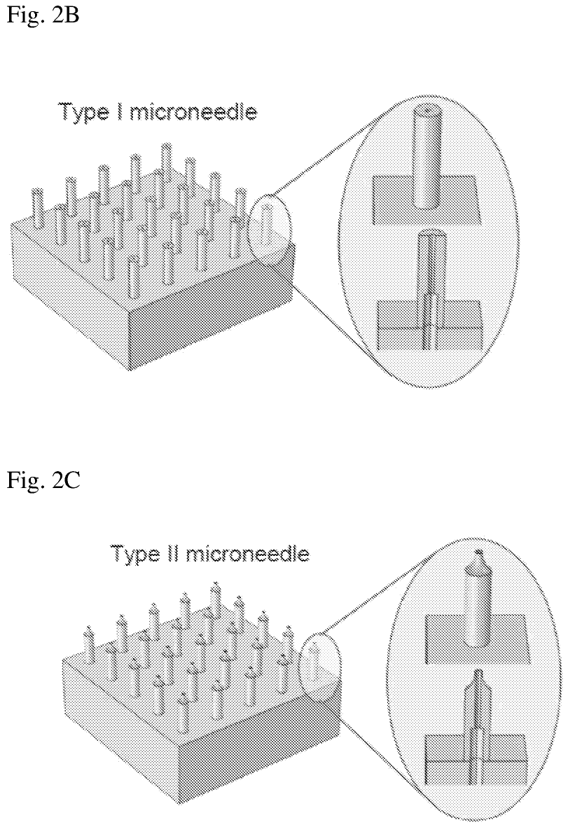

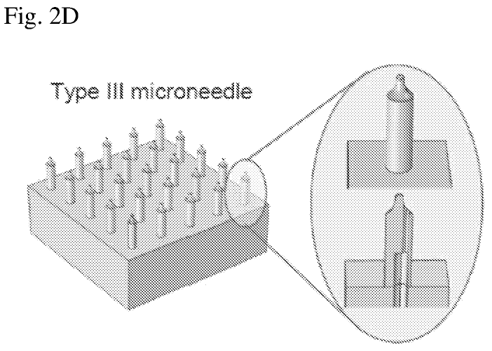

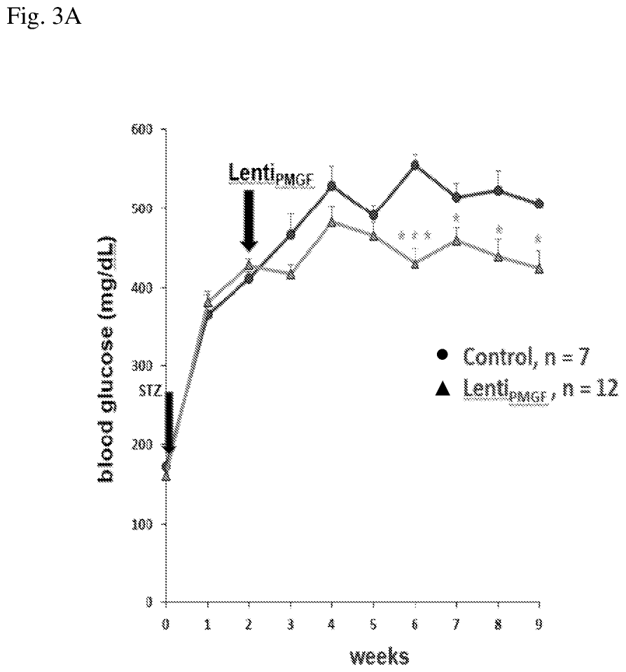

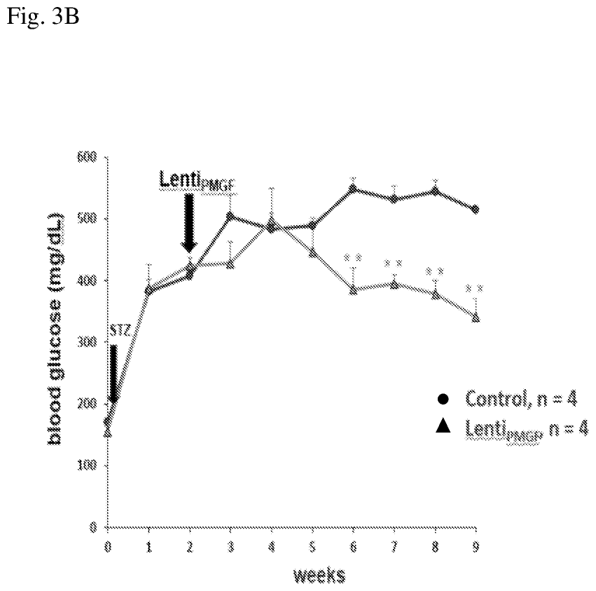

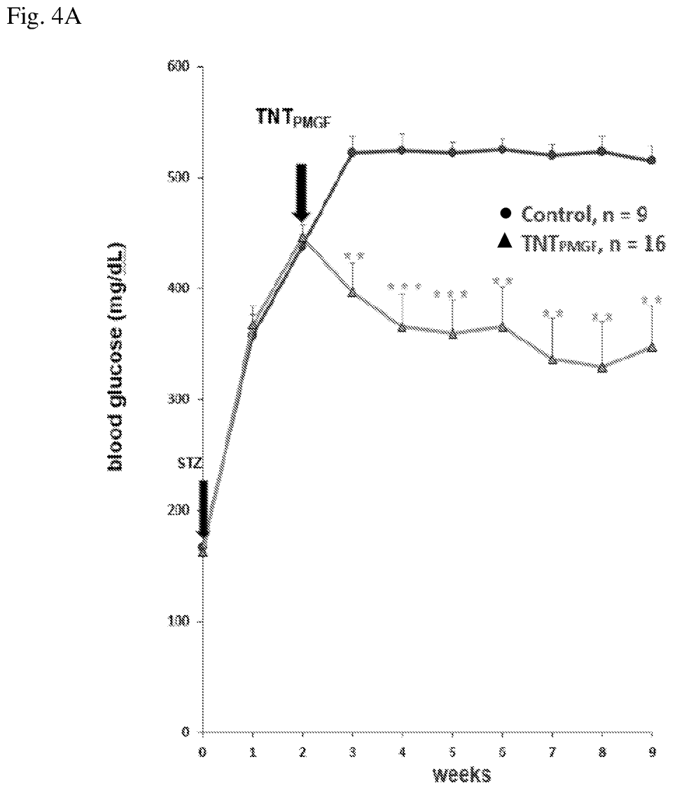

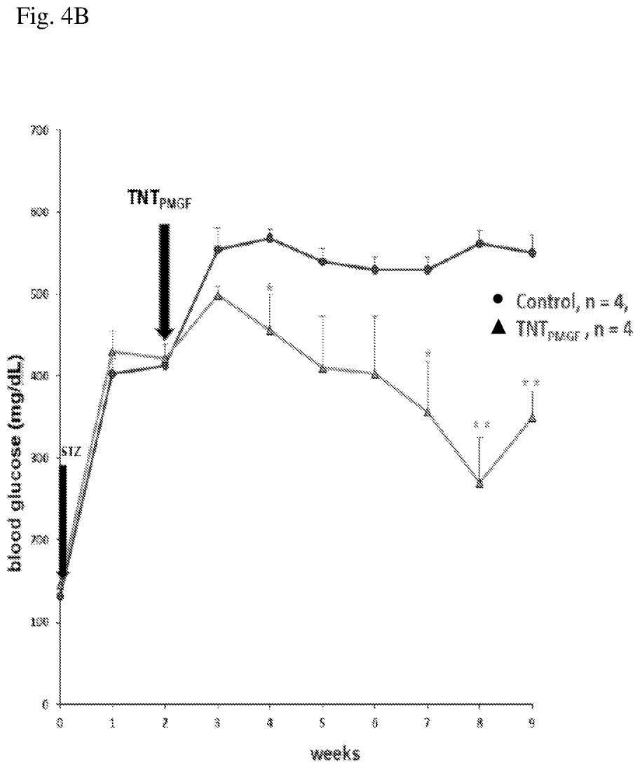

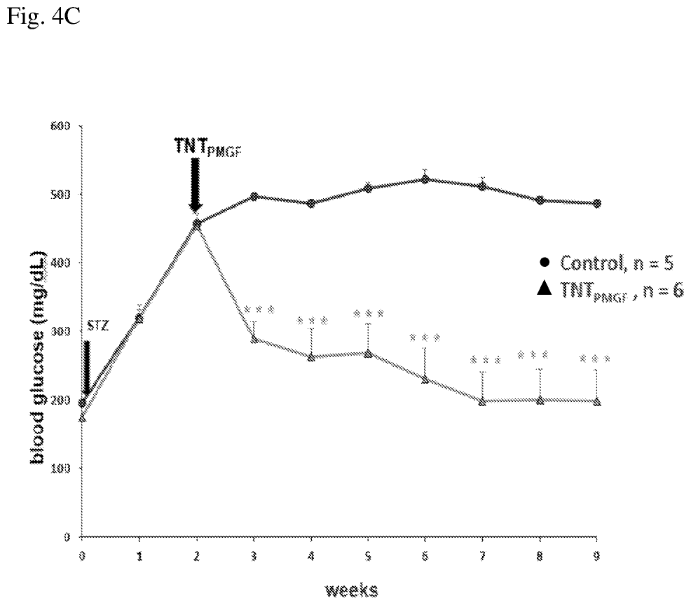

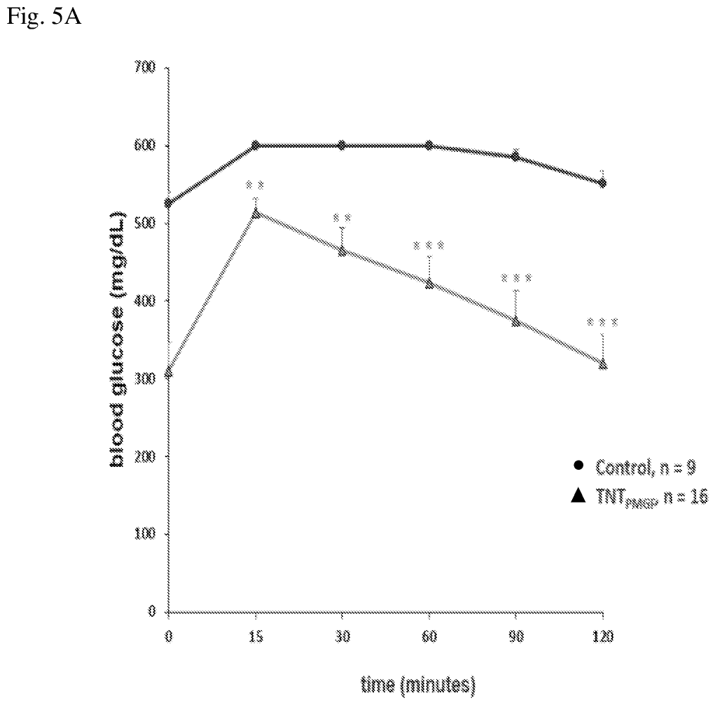

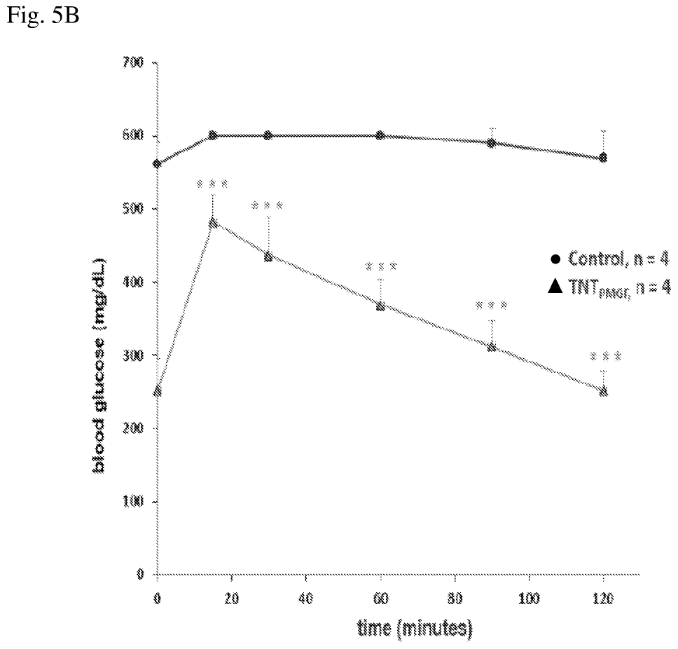

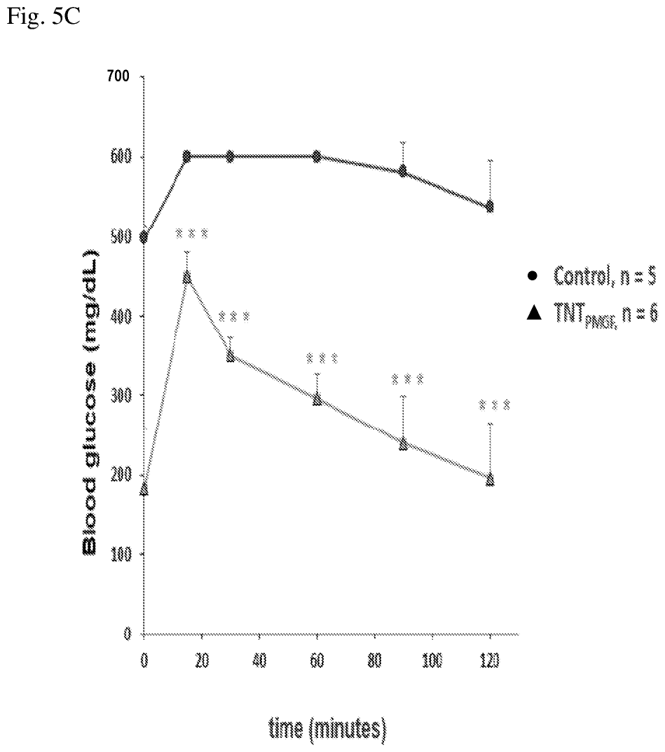

Electroporation is a technique in which an electrical field is applied to cells in order to increase permeability of the cell membrane, allowing cargo (e.g., reprogramming factors) to be introduced into cells (see FIG. 1). Electroporation is a common technique for introducing foreign DNA into cells. FIG. 2A-2D provide examples of microchannel and microneedle arrays that can be used to transfect somatic cells in vivo. Additional details regarding such devices have been described in U.S. patent application nos. 62/903,298 and 62/877,060, the disclosures of which are expressly incorporated by reference.

Tissue nanotransfection allows for direct cytosolic delivery of cargo (e.g, reprogramming factors) into cells by applying a highly intense and focused electric field through arrayed nanochannels, which benignly nanoporates the juxtaposing tissue cell members, and electrophoretically drives cargo into the cells.

In one embodiment, the disclosed compositions are administered in a dose equivalent to parenteral administration of about 0.1 ng to about 100 g per kg of body weight, about 10 ng to about 50 g per kg of body weight, about 100 ng to about 1 g per kg of body weight, from about 1 μg to about 50 mg per kg of body weight, from about 1 mg to about 500 mg per kg of body weight; and from about 1 mg to about 50 mg per kg of body weight. Alternatively, the amount of the disclosed compositions administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

To express a polypeptide or functional nucleic acid, the nucleotide coding sequence may be inserted into appropriate expression vector. Therefore, also disclosed is a non-viral vector comprising a polynucleotide comprising three or more nucleic acid sequences encoding the proteins selected from the group consisting of PDX-1, MafA, GLP1R and FGF21, wherein the three or more nucleic acid sequences are operably linked to an expression control sequence. In some embodiments, the nucleic acid sequences are operably linked to a single expression control sequence, and each coding sequence is preceded with a eukaryotic internal ribosome entry site. In other embodiments, the nucleic acid sequences are operably linked to three or more separate expression control sequences. In some embodiments, the non-viral vector comprises a plasmid.

Methods to construct expression vectors containing genetic sequences and appropriate transcriptional and translational control elements are well known in the art. These methods include in vitro recombinant DNA techniques, synthetic techniques, and in vivo genetic recombination. Such techniques are described in Sambrook et al, Molecular Cloning, A Laboratory Manual (Cold Spring Harbor Press, Plainview, N.Y., 1989), and Ausubel et al., Current Protocols in Molecular Biology (John Wiley & Sons, New York, N.Y., 1989).

In some embodiments, the nucleic acid sequences encoding PDX-1, MafA, GLP1R and, optionally, FGF21 are each separately linked to a eukaryotic expression control sequences, optionally wherein the each of the nucleic acid sequences encoding PDX-1, MafA, GLP1R and FGF21 are linked to a heterologous eukaryotic promoter.

In one embodiment, internal ribosome entry sites (IRES) elements are used to create multigene, or polycistronic, constructs. IRES elements are able to bypass the ribosome scanning model of 5′ methylated Cap dependent translation and begin translation at internal sites. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Disclosed are non-viral vectors containing one or more polynucleotides disclosed herein operably linked to an expression control sequence. Examples of such non-viral vectors include the oligonucleotide alone or in combination with a suitable protein, polysaccharide or lipid formulation. Non-viral methods present certain advantages over viral methods, with simple large scale production and low host immunogenicity being just two. Previously, low levels of transfection and expression of the gene held non-viral methods at a disadvantage; however, recent advances in vector technology have yielded molecules and techniques with transfection efficiencies similar to those of viruses. Examples of suitable non-viral vectors are known by those skilled in the art.

In one embodiment nucleic acids encoding PDX-1, MafA, GLP1R and FGF21 are delivered into the cytosol of a cell in the absence of a delivery vehicle. In one embodiment electroporation is used to stimulate uptake of nucleic acids encoding PDX-1, MafA, GLP1R and FGF21.

The compositions disclosed herein can be used therapeutically in combination with a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic.

Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the nucleic acids, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines

The herein disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeal, ophthalmically, vaginally, rectally, intranasally, topically or the like, including topical intranasal administration or administration by inhalant.

In accordance with one embodiment somatic cells of a patient are reprogrammed to be insulinogenic by enhancing the intracellular concentration of the proteins PDX1, MafA, GLP1R and FGF21 in the target tissue. Intracellular concentrations of PDX1, MafA, GLP1R and FGF21 can be enhanced using any of the standard molecular biological techniques known to those skilled in the art. In one embodiment, intracellular concentrations of PDX1, MafA, GLP1R and FGF21 can be enhanced by the introduction of regulatory elements into the respective native PMGF gene (e.g., a heterologous promoter or enhancer element) or by introducing other factors such as a gene silencer or epigenetic manipulators that target DNA demethylation and chromatin remodeling. In one embodiment the native genes encoding the respective PMGF proteins are modified to enhance their expression using standard gene editing techniques, including for example the use of CRISPR technology. Alternatively, enhancing the intracellular concentration of PDX1, MafA, GLP1R and FGF21 polypeptide can also be achieved by the introduction of exogenous components (e.g. proteins and nucleic acids) into the cytosol of skin cells wherein the exogenous components directly or indirectly enhance the intracellular concentration of PDX1, MafA, GLP1R and FGF21. In one embodiment the introduced exogenous components comprise nucleic acid sequences (e.g., DNA, mRNA, miRNA and RNAi) that enhance the expression of genes encoding for the PDX1, MafA, GLP1R and FGF21 polypeptides. In one embodiment the exogenous component introduced into the cell are DNAs that encodes for each of the PDX1, MafA, GLP1R and FGF21 polypeptides.

In accordance with the present invention nucleic acid and/or proteins are introduced into the cytosol of post-natal somatic cells such as skin cells to induce reprogramming of the target cells. Any of the standard techniques for introducing macromolecules into cells can be used in accordance with the present invention. Known delivery methods can be broadly classified into two types. In the first type, a membrane-disruption-based method involving mechanical, thermal or electrical means can be used to disrupt the continuity of the cell membrane with enhanced permeabilization for direct penetration of desired macromolecules. In the second type, a carrier-based method, using various viruses, exosomes, vesicles and nanoparticle capsules, allows uptake of the carrier through endocytosis and fusion processes of cells for delivery of the carrier payload.

Among the methods of permeabilization-based disruption delivery, electroporation has already been established as a universal tool. High efficiency delivery can be achieved with minimum cell toxicity by careful control of the electric field distribution. In accordance with one embodiment nucleic acid sequences encoding for PDX1, MafA, GLP1R and FGF21 polypeptides are delivered to the cytosol of somatic cells through the use of tissue nanotransfection (TNT).

Tissue nanotransfection (TNT) is an electromotive gene transfer technology that delivers plasmids, RNA and oligonucleotides to live tissue causing direct conversion of tissue function in vivo under immune surveillance without the need for any laboratory procedures. Unlike viral gene transfer commonly used for in vivo tissue reprogramming, TNT obviates the need for a viral delivery vehicle and thus minimizes the risk of genomic integration or cell transformation.

Current methods of in vivo reprogramming can involve transfecting cells in vivo, or in vitro followed by implantation. Although one embodiment of the present invention entails in vitro reprogramming of cells followed by transplantation, cell implants are often met with low survival and poor tissue integration. Additionally, transfecting cells in vitro involves additional regulatory and laboratory hurdles.

In accordance with one embodiment somatic cells are transfected in vivo with a reprogramming cocktail as disclosed herein. Common methods for bulk in vivo transfection are delivery of viral delivery vehicles, non-viral delivery vehicles or electroporation. Although viral vectors can be used in accordance with the present disclosure for delivery of a reprogramming cocktail to non-pancreatic somatic cells, viral vectors suffer the drawback of potentially initiating undesired immune reactions. In addition, many viral vectors cause long term expression of gene, which is useful for some applications of gene therapy, but for applications where sustained gene expression is unnecessary or even undesired, transient transfection is a viable option. Viral vectors also involve insertional mutagenesis and genomic integration that can have undesired side effects. However, in accordance with one embodiment certain non-viral carriers, such as liposomes or exosomes can be used to deliver a reprogramming cocktail to somatic cells in vivo.

TNT provides a method for localized gene delivery that causes direct conversion of tissue function in vivo under immune surveillance without the need for any laboratory procedures. By using TNT with plasmids, it is possible to temporally and spatially control overexpression of a gene. Spatial control with TNT allows for transfection of a target area such as a portion of skin tissue without transfection of other tissues.

As disclosed in greater detail in the Examples, a hollow needle array structure has been designed that enables efficient cutaneous delivery of loaded drugs including nucleic acid sequences. Three different types of silicon hollow needle arrays can be prepared for TNT applications (as shown schematically in FIGS. 2B-2D) with bore diameter ranging from nm to μm in sizes. In less than a second, the silicon hollow needle arrays disclosed herein enable delivery of active factors to specific depth in mouse, rat and human tissue.

In accordance with one embodiment a composition is provided for reprogramming cells and tissues, and more particularly reprogramming skin tissues in vivo. In one embodiment the composition comprises a first nucleic acid sequence encoding for Pancreatic And Duodenal Homeobox 1 (PDX-1); a second nucleic acid sequence encoding for transcription factor MafA; a third nucleic acid sequence encoding for glucagon-like peptide 1 receptor (GLP-1R); and optionally a fourth nucleic acid sequence comprising nucleic acid sequence encoding for Fibroblast growth factor 21 (FGF21), wherein each of said first, second, third and optional fourth nucleic acid sequences are operably linked to regulatory sequences for expression of the encoded proteins in eukaryotic cells, including mammalian cells. In one embodiment the composition comprises each of the first, second, third and fourth nucleic acid sequences. In one embodiment the composition consists of the first, second, third and fourth nucleic acid sequences and a pharmaceutically acceptable carrier, optionally wherein each of the first, second, third and fourth nucleic acid sequences are operably linked to a heterologous promoter.

In accordance with one embodiment a reprogramming cocktail solution is provided wherein the solution comprises a first nucleic acid sequence that encodes a peptide having at least 80%, 85%, 95% or 99% sequence identity to SEQ ID NO: 2; a second nucleic acid sequence that encodes a peptide having at least 80%, 85%, 95% or 99% sequence identity to SEQ ID NO: 4; a third nucleic acid sequence that encodes a peptide having at least 80%, 85%, 95% or 99% sequence identity to SEQ ID NO: 6; and an optional fourth nucleic acid sequence encoding a peptide having at least 80%, 85%, 95% or 99% sequence identity to SEQ ID NO: 8. In one embodiment the reprogramming cocktail solution comprises purified or isolated nucleic acid sequences that encode the proteins of SEQ ID NOs 2, 4, 6 and 8.

In accordance with one embodiment a reprogramming cocktail solution is provided wherein the solution comprises a first nucleic acid sequence that encodes a peptide having at least 80%, 85%, 95% or 99% sequence identity to SEQ ID NO: 2; a second nucleic acid sequence that encodes a peptide having at least 80%, 85%, 95% or 99% 95% sequence identity to SEQ ID NO: 4; a third nucleic acid sequence that encodes a peptide having at least 80%, 85%, 95% or 99% sequence identity to SEQ ID NO: 6; and a fourth nucleic acid sequence encoding a peptide having at least 80%, 85%, 95% or 99% sequence identity to SEQ ID NO: 8.

In accordance with one embodiment a reprogramming cocktail solution is provided wherein the solution comprises a first nucleic acid sequence that encodes a peptide of SEQ ID NO: 2; a second nucleic acid sequence that encodes a peptide of SEQ ID NO: 4; a third nucleic acid sequence that encodes a peptide of SEQ ID NO: 6; and a fourth nucleic acid sequence encoding a peptide of SEQ ID NO: 8, optionally wherein each of the first, second, third and fourth nucleic acid sequences are operably linked to a heterologous promoter.

In accordance with one embodiment the reprogramming cocktail solution comprises multiple non-viral expression vectors that comprise the first second, third and fourth nucleic acid sequences. In one embodiment the reprogramming cocktail solution comprises four distinct plasmids each comprising one of the first, second, third and fourth nucleic acid sequences operably linked to a promoter, as well as other regulatory sequences, that enable expression of the encoded proteins within eukaryotic cells. In one embodiment two or more of said first, second, third and fourth nucleic acids are located on an expression vector, wherein the expression vector comprises a single promoter operably linked to a multiple coding sequence, wherein the multiple coding sequence comprises said two or more of the first, second, third and fourth nucleic acid sequences wherein internal ribosome entry sites are present before each of said two or more first, second, third and fourth nucleic acid sequences.

In one embodiment the reprogramming cocktail solution comprises only one distinct type of plasmid/expression vector wherein the plasmid/expression vector comprises all four of the first, second, third and fourth nucleic acid sequences linked together to form a multiple coding sequence wherein the multiple coding sequence comprises all four of said first, second, third and fourth nucleic acid sequence, each proceeded by an internal ribosome entry sites and all are operably linked to said single promoter that is operable in a mammalian cell. In one embodiment the plasmid/expression vector is a non-viral expression vector. In accordance with one embodiment the reprogramming cocktail solution further comprises a reagent that enhances electroporation efficiency of delivering nucleic acids into the interior of a eukaryotic cell or mammalian tissue.

One embodiment of the present disclosure is directed to a polynucleotide comprising three or more nucleic acid sequences encoding transcription factors/proteins selected from the group consisting of PDX-1, MafA, GLP1R, and, optionally, FGF21. The PDX-1, MafA, GLP1R, and FGF21 proteins may be mammalian proteins, such as human proteins. In one embodiment the encoded PDX-1, MafA, GLP1R, and FGF21 proteins comprise the amino acid sequence of SEQ ID NO: 2, SEQ ID NO: 4, SEQ ID NO: 6 and SEQ ID NO: 8, respectively, or peptides that differ from the amino acid sequence of SEQ ID NO: 2, SEQ ID NO: 4, SEQ ID NO: 6 and SEQ ID NO: 8, by 1-10, 1-5 or 1-3 amino acid substitutions, insertions or deletions, or peptides that differ from the amino acid sequence of SEQ ID NO: 2, SEQ ID NO: 4, SEQ ID NO: 6 and SEQ ID NO: 8, by 1-10, 1-5 or 1-3 amino acid substitutions, optionally conservative amino acid substitutions.

In one embodiment a reprogramming cocktail solution is provided comprising a non-viral vector, wherein the vector comprises a polynucleotide comprising three or more nucleic acid sequences encoding proteins selected from the group consisting of PDX-1, MafA, GLP1R, and FGF21, where the three or more nucleic acid sequences are operably linked to an expression control sequences. Each of the nucleic acid sequences may be individually operably linked to a single promoter and other regulatory sequences required for expression in eukaryotic cells, or alternatively multiple nucleic acid sequences can be expressed under the control of a single promoter.

In accordance with one embodiment a reprogramming cocktail solution comprises peptides, more particularly in one embodiment a composition is provided comprising
a peptide having at least 95% sequence identity to SEQ ID NO: 2;
a peptide having at least 95% sequence identity to SEQ ID NO: 4;
a peptide having at least 95% sequence identity to SEQ ID NO: 6;
a peptide having at least 95% sequence identity to SEQ ID NO: 8; and optionally a reagent that enhances efficiency of delivering proteins into the interior of a eukaryotic cell.

A further embodiment is directed to a method to reprogram a somatic cell to an insulinogenic cell, optionally having insulinogenic characteristics of a pancreatic β-cell (i.e. a pancreatic β-like cell) by (a) delivering intracellularly into the somatic cell the proteins PDX-1, MafA, GLP1R, and optionally FGF21, or polynucleotides encoding the proteins PDX-1, MafA, GLP1R, and optionally FGF21 proteins. In one embodiment the somatic cell is a skin cell, and more particularly the transfected cells are skin cells of skin tissue transfected in vivo with the reprogramming cocktail solution, and optionally in the absence of a viral delivery vehicle. In one embodiment the PDX-1 protein, MafA protein, and GLP1R protein and optionally the FGF21 protein, or a polynucleotide encoding the PDX-1 protein, MafA protein GLP1R protein and optionally the FGF21 protein are delivered intracellularly using any standard technique known to those skilled in the art. In one embodiment intracellular delivery is via a viral vector, or other delivery vehicle capable of interacting with a cell membrane to deliver its contents into a cell. In one embodiment intracellular delivery is via three-dimensional nanochannel electroporation, delivery by a tissue nanotransfection device, or delivery by a deep-topical tissue nanoelectroinjection device. In one embodiment the reprogramming cocktail is delivered into the cytosol of post-natal skin tissue cells in vivo through tissue nanotransfection (TNT) using a silicon hollow needle array.

In one embodiment a method of reprograming a non-pancreatic somatic tissue, optionally reprogramming the cells of post-natal skin tissue in vivo, to produce insulin and C-peptide comprises the step of: delivering intracellularly into said non-pancreatic somatic tissue any of the reprogramming cocktail solutions of the present disclosure, optionally by TNT. In one embodiment the reprogramming cocktail solution comprises or consists of naked DNA, wherein the naked DNA comprises a first nucleic acid sequence encoding a peptide having at least 95% sequence identity to SEQ ID NO: 2, a second nucleic acid sequence encoding a peptide having at least 95% sequence identity to SEQ ID NO: 4; a third nucleic acid sequence encoding a peptide having at least 95% sequence identity to SEQ ID NO: 6; and optionally a fourth nucleic acid sequence encoding a peptide having at least 95% sequence identity to SEQ ID NO: 8. In one embodiment the reprogramming cocktail solution comprises or consists of naked DNA, wherein the naked DNA comprises each of the first, second, third and fourth nucleic acids. Any of the first, second, third and fourth nucleic acid sequences disclosed herein can be located on separate plasmids or expression vectors or can be clustered together in groups on individual plasmids or expression vectors. In one embodiment each of the first, second, third and fourth nucleic acid sequences are all located on a single plasmid or expression vectors as separate genes under the control of individual promoters or as a single multigene construct under the control of a single promoter.

In one embodiment the reprogramming cocktail solution comprises one or more distinct expression vectors wherein each of the expression vectors comprises two or more of said first, second, third and fourth nucleic acids are part of an expression vector wherein the expression vector comprises a single eukaryotic promoter operably linked to a multiple coding sequence that comprises said two or more first, second, third and fourth nucleic acid sequences wherein said multiple coding sequence further comprises internal ribosome entry sites present before each of said two or more first, second, third and fourth nucleic acid sequences. In one embodiment each of said first, second, third and fourth nucleic acids are located on a single expression vector as part of a multiple coding sequence, and the multiple coding sequence further comprises internal ribosome entry sites present before each of said two or more first, second, third and fourth nucleic acid sequences and a single promoter driving the transcription of the multiple coding sequence.

In one embodiment a method of normalizing blood glucose levels a subject with diabetes is provided wherein the method comprises the step of reprogramming targeted post-natal skin tissue in vivo to produce insulin. In one embodiment the method comprises delivering any of the reprogramming cocktail solutions of the present disclosure into the cytosol of cells of the target skin tissue. Any of the known techniques for transfecting cells can be used, including TNT. In one embodiment the reprogramming cocktail solution comprises a first nucleic acid sequence encoding a peptide having at least 95% sequence identity to SEQ ID NO: 2, a second nucleic acid sequence encoding a peptide having at least 95% sequence identity to SEQ ID NO: 4; and a third nucleic acid sequence encoding a peptide having at least 95% sequence identity to SEQ ID NO: 6; and optionally a fourth nucleic acid sequence encoding a peptide having at least 95% sequence identity to SEQ ID NO: 8. In one embodiment the reprogramming cocktail solution comprises a first nucleic acid sequence having at least 95% sequence identity to SEQ ID NO: 1, a second nucleic acid sequence having at least 95% sequence identity to SEQ ID NO: 3; a third nucleic acid sequence having at least 95% sequence identity to SEQ ID NO: 5; and optionally a fourth nucleic acid sequence having at least 95% sequence identity to SEQ ID NO: 7. In one embodiment the reprogramming cocktail solution comprises a first nucleic acid sequence having at least 95% sequence identity to SEQ ID NO: 1, a second nucleic acid sequence having at least 95% sequence identity to SEQ ID NO: 3; a third nucleic acid sequence having at least 95% sequence identity to SEQ ID NO: 5; and a fourth nucleic acid sequence having at least 95% sequence identity to SEQ ID NO: 7.

In one embodiment a method is provided for treating diabetic or pre-diabetic patients by direct tissue reprogramming of somatic tissue (i.e., skin or fat or another non-pancreatic somatic tissue or pancreatic somatic tissue) to convert the somatic cells to insulinogenic cells, optionally where the reprogrammed cells have characteristics of a pancreatic β-cell (i.e. a pancreatic β-like cell). The reprogrammed cells produced by the methods as disclosed herein secrete at least 15%, or at least 25% or at least 30% of the insulin that endogenous β-cells secrete, or alternatively, in some embodiments, the reprogrammed cells exhibits at least two characteristics of an endogenous pancreatic β-cell such as secreting insulin and becoming positive for applicable biomarkers including the detection of insulin C-peptide.

In accordance with one embodiment a method for treating Type 1 or Type2 diabetes and/or moderating blood glucose levels towards normal levels (i.e., between 70 and 100 mg/dL) is provided wherein somatic tissues of a patient are induced to have elevated intracellular concentrations of the polypeptides Pancreatic And Duodenal Homeobox 1 (PDX-1), transcription factor MafA, glucagon-like peptide 1 receptor (GLP-1R); and Fibroblast growth factor 21 (FGF21). In one embodiment increased levels of those polypeptides is achieved by introducing a first nucleic acid sequence encoding for Pancreatic And Duodenal Homeobox 1 (PDX-1), a second nucleic acid sequence encoding for transcription factor MafA, a third nucleic acid sequence encoding for glucagon-like peptide 1 receptor (GLP-1R); and a fourth nucleic acid sequence comprising nucleic acid sequence encoding for Fibroblast growth factor 21 (FGF21) into the cytosol of skin cells in vivo. In one embodiment the nucleic acid sequences are introduced into the cytosol of cells of the target tissue via nanotransfection (TNT).

In accordance with one embodiment a kit is provided for conducting in vivo transfection of post-natal skin tissue and inducing the skin tissue to become insulinogenic and optionally exhibit characteristics of a pancreatic β-cell. In one embodiment the kit comprises a disposable nanotransfection device and a reprogramming cocktail. In one embodiment the nanotransfection device comprises a silicon wafer comprising a series of microchannels. In one embodiment the nanotransfection device comprises a plurality of shafts, wherein each of the plurality of shafts has an exterior surface that is electrically conductive, wherein each of the plurality of shafts is electrically coupled to each other of the plurality of shafts, wherein each of the plurality of shafts extend from a proximal end to a distal end, each of the plurality of shafts defining a primary channel interior to the corresponding shaft extending from the proximal end toward the distal end, wherein the primary channel is open at the proximal end and closed at the distal end, wherein each of the plurality of shafts further defines one or more microchannels, wherein each of the one or more microchannels extends from the primary channel through a wall of the corresponding shaft, wherein each of the one or more microchannels has a diameter less than 10 micrometers. The kit may further comprise a plurality of electrodes, wherein each of the plurality of electrodes is electrically coupled to each other of the plurality of electrodes, wherein the plurality of electrodes are disposed adjacent to the plurality of shafts such that, when a voltage is applied between the plurality of shafts and the plurality of electrodes, an electric field is created perpendicular to an axis of each of the plurality of shafts.

In accordance with one embodiment a kit is provided for conducting in vivo transfection of post-natal skin tissue and inducing the skin tissue to become insulinogenic wherein the kit comprises a disposable nanotransfection device and a reprogramming cocktail, wherein the nanotransfection device comprises a hollow microneedle array with one or more compartments for receiving a reprogramming cocktail solution. In one embodiment the nanotransfection device is selected from the group consisting of a type I Type I hollow microneedle array with flat tip, a Type II hollow microneedle array with sharp tip and centered bore, and a Type III hollow microneedle array with sharp tip and off-centered bore as shown in FIGS. 2B-2D. In one embodiment length of a cylindrical needle of the Type I, II and III microneedle arrays is about 210 μm, with the outer diameter being about 50 μm, and the diameter of the hollow channel located at the center of the needle is about 6 μm. The spacing between two adjacent needles is about 150 μm. In one embodiment the diameter of the backside hole is about 20 μm and the spacing is the same as the hollow microchannels. Type II and type III microneedles are expected to have the similar delivery result, but with additional functionality. Different from the flat tip, the sharpness of type II needle arrays make for better performance in reducing the insertion force required for insertion into the tissue. The type III silicon hollow needle arrays shown in FIG. 2D have a sharp tip and off-center bore. The hollow bore is designed with a deviation of about 15 μm from the center of the needle to decrease the incidence of tissue clogging during insertion.

In one embodiment the hollow microneedle array comprises an electrode (i.e., cathode, optionally gold-coated or silver-coated) that is positioned for contact with a solution loaded into the compartment of the device and a needle counter-electrode (i.e., anode) positioned for insertion intradermally on a patient's skin. In one embodiment the reprogramming cocktail solution comprises a first nucleic acid sequence encoding for Pancreatic And Duodenal Homeobox 1 (PDX-1), a second nucleic acid sequence encoding for transcription factor MafA, a third nucleic acid sequence encoding for glucagon-like peptide 1 receptor (GLP-1R); and optionally a fourth nucleic acid sequence comprising nucleic acid sequence encoding for Fibroblast growth factor 21 (FGF21). In accordance with one embodiment the nanotransfection device is preloaded with the reprogramming cocktail solution.

In accordance with embodiment 1, a method of reprograming post neonatal cells of a somatic tissue to produce insulin and C-peptide is provided, wherein the method comprises the step of delivering intracellularly into said cells of the somatic tissue, optionally in the absence of a viral delivery vehicle, DNA comprising a first nucleic acid sequence encoding a peptide having at least 95% sequence identity to SEQ ID NO: 2;

a second nucleic acid sequence encoding a peptide having at least 95% sequence identity to SEQ ID NO: 4;

a third nucleic acid sequence encoding a peptide having at least 95% sequence identity to SEQ ID NO: 6; and optionally

a fourth nucleic acid sequence encoding a peptide having at least 95% sequence identity to SEQ ID NO: 8.

In accordance with embodiment 2 the method of embodiment 1 is provided wherein said first, second, third and fourth nucleic acid sequences are each delivered simultaneously into the cytosol of cells of said somatic tissue in vivo.

In accordance with embodiment 3 the method of embodiment 1 or 2 is provided wherein one or more expression vectors are transfected into said cells of the somatic tissue wherein said expression vectors comprise said first, second, third and fourth nucleic acid sequences.

In accordance with embodiment 4 the method of any one of embodiments 1-3 is provided wherein two or more of said first, second, third and fourth nucleic acids are part of an expression vector wherein the expression vector comprises a single eukaryotic promoter operably linked to a multiple coding sequence that comprises said two or more first, second, third and fourth nucleic acid sequences wherein said multiple coding sequence further comprises internal ribosome entry sites present before each of said two or more first, second, third and fourth nucleic acid sequences, optionally wherein said first nucleic acid sequence comprises a sequence encoding a peptide having at least 95%, 99% or 100% sequence identity to SEQ ID NO: 2; said second nucleic acid sequence comprises a sequence encoding a peptide having at least 95%, 99% or 100% sequence identity to SEQ ID NO: 4; said third nucleic acid sequence comprising a sequence encoding a peptide having at least 95%, 99% or 100% sequence identity to SEQ ID NO: 6; and said fourth nucleic acid sequence comprising a sequence encoding a peptide having at least 95%, 99% or 100% sequence identity to SEQ ID NO: 8; optionally wherein said first nucleic acid sequence comprises a sequence of SEQ ID NO: 1, said second nucleic acid sequence comprises a sequence of SEQ ID NO: 3, said third nucleic acid sequence comprises a sequence of SEQ ID NO: 5 and said fourth nucleic acid sequences comprises a sequence of SEQ ID NO: 7.

In accordance with embodiment 5 the method of any one of embodiments 1˜4 is provided wherein each of said first, second, third and fourth nucleic acids are located on a single expression vector, optionally wherein said expression vector comprises a single eukaryotic promoter operably linked to a multiple coding sequence comprising each of said first, second, third and fourth nucleic acids, wherein an internal ribosome entry sites is present before each of said first, second, third and fourth nucleic acid sequences.

In accordance with embodiment 6 the method of any one of embodiments 1-5 is provided wherein the somatic cell is a skin cell.

In accordance with embodiment 7 the method of any one of embodiments 1-6 is provided wherein the intracellular delivery is via tissue nanotransfection.

In accordance with embodiment 8 the method of any one of embodiments 1-7 is provided wherein the cells are skin cells of skin tissue transfected in vivo.

In accordance with embodiment 9 a method of reducing blood glucose levels towards normalize levels in a subject with diabetes is provided, wherein the method comprises the step of reprogramming targeted skin tissue in vivo to produce insulin, said reprogramming step comprising contacting the cells of target skin tissue with a reprogramming composition under conditions that enhance cellular uptake of the reprogramming composition components, wherein the reprogramming composition comprises a first nucleic acid sequence encoding a peptide having at least 95%, 99% or 100% sequence identity to SEQ ID NO: 2; a second nucleic acid sequence encoding a peptide having at least 95%, 99% or 100% sequence identity to SEQ ID NO: 4; and a third nucleic acid sequence encoding a peptide having at least 95%, 99% or 100% sequence identity to SEQ ID NO: 6; and optionally a fourth nucleic acid sequence encoding a peptide having at least 95%, 99% or 100% sequence identity to SEQ ID NO: 8 to said target skin cells.

In accordance with embodiment 10 a composition for use in reprograming post neonatal cells of a somatic tissue to produce insulin and C-peptide is provided. In one embodiment the composition comprises

a first nucleic acid sequence encoding for Pancreatic And Duodenal Homeobox 1 (PDX-1), optionally wherein the first nucleic acid sequence encodes a peptide having at least 95%, 99% or 100% sequence identity to SEQ ID NO: 2;

a second nucleic acid sequence encoding for transcription factor MafA, optionally wherein said second nucleic acid sequence encodes a peptide having at least 95%, 99% or 100% sequence identity to SEQ ID NO: 4;

a third nucleic acid sequence encoding for glucagon-like peptide 1 receptor (GLP-1R), optionally wherein said third nucleic acid sequence encodes a peptide having at least 95%, 99% or 100% sequence identity to SEQ ID NO: 6; and optionally

a fourth nucleic acid sequence comprising nucleic acid sequence encoding for Fibroblast growth factor 21 (FGF21) optionally wherein said fourth nucleic acid sequence encodes a peptide having at least 95%, 99% or 100% sequence identity to SEQ ID NO: 8, wherein each of said first, second, third and optional fourth nucleic acid sequences are operably linked to eukaryotic regulatory sequences.

In accordance with embodiment 11 the composition of embodiment 10 is provided wherein the first nucleic acid sequence encodes a peptide having at least 95% sequence identity to SEQ ID NO: 2;

the second nucleic acid sequence encodes a peptide having at least 95% sequence identity to SEQ ID NO: 4;

the third nucleic acid sequence encodes a peptide having at least 95% sequence identity to SEQ ID NO: 6; and said optional a fourth nucleic acid sequence encodes a peptide having at least 95% sequence identity to SEQ ID NO: 8.

In accordance with embodiment 12 the composition of embodiment 10 or 11 is provided wherein said composition comprises said first, second, third and fourth nucleic acid sequences.

In accordance with embodiment 13 the composition of any one of embodiments 10-12 is provided wherein two or more of said first, second, third and fourth nucleic acids are part of an expression vector wherein the expression vector comprises a single eukaryotic promoter operably linked to a multiple coding sequence that comprises said two or more first, second, third and fourth nucleic acid sequences wherein said multiple coding sequence further comprises internal ribosome entry sites present before each of said two or more first, second, third and fourth nucleic acid sequences.

In accordance with embodiment 14 the composition of any one of embodiments 10-13 is provided wherein said multiple coding sequence comprises all four of said first, second, third and optionally fourth nucleic acid sequence, each proceeded by an internal ribosome entry sites and operably linked to said single eukaryotic promoter.

In accordance with embodiment 15 the composition of any one of embodiments 10-14 is provided wherein the first, second, third and fourth nucleic acid sequences are part of a non-viral vector.

In accordance with embodiment 16 a kit for conducting in vivo transfection of post-natal skin tissue and inducing the skin tissue to be insulinogenic is provided, wherein said kit comprises
a disposable nanotransfection device; and
a reprogramming cocktail, wherein the reprogramming cocktail solution comprises a first nucleic acid sequence encoding for Pancreatic And Duodenal Homeobox 1 (PDX-1), a second nucleic acid sequence encoding for transcription factor MafA, a third nucleic acid sequence encoding for glucagon-like peptide 1 receptor (GLP-1R); and a fourth nucleic acid sequence comprising nucleic acid sequence encoding for Fibroblast growth factor 21 (FGF21).

In accordance with embodiment 17 the kit of embodiment 16 is provided wherein the nanotransfection device comprises a hollow microneedle array with one or more compartments for receiving said reprogramming cocktail solution.

### EMBODIMENTS

In accordance with embodiment 1 a method of reprograming cells of a somatic tissue to produce insulin and C-peptide is provided, said method comprising the step of:

delivering intracellularly into said cells of the somatic tissue DNA comprising
a first nucleic acid sequence encoding a peptide having at least 95% sequence identity to SEQ ID NO: 2;
a second nucleic acid sequence encoding a peptide having at least 95% sequence identity to SEQ ID NO: 4;
a third nucleic acid sequence encoding a peptide having at least 95% sequence identity to SEQ ID NO: 6; and optionally
a fourth nucleic acid sequence encoding a peptide having at least 95% sequence identity to SEQ ID NO: 8.

In accordance with embodiment 2, the method of embodiment 1 is provided wherein said first, second, third and fourth nucleic acid sequences are each delivered simultaneously into the cytosol of cells of said somatic tissue in vivo.

In accordance with embodiment 3, the method of embodiment 1 or 2 is provided wherein one or more expression vectors are transfected into said cells of the somatic tissue wherein said expression vectors comprise said first, second, third and fourth nucleic acid sequences.

In accordance with embodiment 4, the method of any one of embodiments 1-3 is provided wherein two or more of said first, second, third and fourth nucleic acids are part of an expression vector wherein the expression vector comprises a single eukaryotic promoter operably linked to a multiple coding sequence that comprises said two or more first, second, third and fourth nucleic acid sequences wherein said multiple coding sequence further comprises internal ribosome entry sites present before each of said two or more first, second, third and fourth nucleic acid sequences.

In accordance with embodiment 5 the method of any one of embodiments 1˜4 is provided wherein each of said first, second, third and fourth nucleic acids are located on a single expression vector.

In accordance with embodiment 6 the method of any one of embodiments 1-5 is provided wherein the somatic cell is a skin cell.

In accordance with embodiment 7 the method of any one of embodiments 1-6 is provided wherein the intracellular delivery is via tissue nanotransfection.

In accordance with embodiment 8 the method of embodiment 7 is provided wherein the cells are skin cells of skin tissue transfected in vivo.

In accordance with embodiment 9 a method of normalizing blood glucose levels in a subject with diabetes is provided, said method comprising the step of reprogramming targeted skin tissue in vivo to produce insulin, said method comprising

contacting the cells of said target skin tissue with a reprogramming composition under conditions that enhance cellular uptake of the reprogramming composition components, wherein the reprogramming composition comprises
a first nucleic acid sequence encoding a peptide having at least 85%, 95% or 99% sequence identity to SEQ ID NO: 2;
a second nucleic acid sequence encoding a peptide having at least 85%, 95% or 99% sequence identity to SEQ ID NO: 4; and
a third nucleic acid sequence encoding a peptide having at least 85%, 95% or 99% sequence identity to SEQ ID NO: 6; and optionally
a fourth nucleic acid sequence encoding a peptide having at least 85%, 95% or 99% sequence identity to SEQ ID NO: 8 to said target skin cells.

In accordance with embodiment 10 the method of embodiment 9 is provided wherein the reprogramming composition comprises

a first nucleic acid sequence encoding a peptide comprising SEQ ID NO: 2;

a second nucleic acid sequence encoding a peptide comprising SEQ ID NO: 4;

a third nucleic acid sequence encoding a peptide comprising SEQ ID NO: 6; and

a fourth nucleic acid sequence encoding a peptide comprising SEQ ID NO: 8

In accordance with embodiment 11 a composition is provided comprising a first nucleic acid sequence encoding for Pancreatic And Duodenal Homeobox 1 (PDX-1);

a second nucleic acid sequence encoding for transcription factor MafA;

a third nucleic acid sequence encoding for glucagon-like peptide 1 receptor (GLP-1R); and optionally

a fourth nucleic acid sequence comprising nucleic acid sequence encoding for Fibroblast growth factor 21 (FGF21), wherein each of said first, second, third and optional fourth nucleic acid sequences are operably linked to eukaryotic regulatory sequences.

In accordance with embodiment 12, the composition of embodiment 11 is provided wherein said first nucleic acid sequence encodes a peptide having at least 85%, 95% or 99% sequence identity to SEQ ID NO: 2;

said second nucleic acid sequence encodes a peptide having at least 85%, 95% or 99% sequence identity to SEQ ID NO: 4; and

said third nucleic acid sequence encodes a peptide having at least 85%, 95% or 99% sequence identity to SEQ ID NO: 6; and optionally

said fourth nucleic acid sequence encodes a peptide having at least 85%, 95% or 99% sequence identity to SEQ ID NO: 8 to said target skin cells.

In accordance with embodiment 13 the method of embodiment 11 or 12 is provided wherein the reprogramming composition comprises

a first nucleic acid sequence encoding a peptide comprising SEQ ID NO: 2;

a second nucleic acid sequence encoding a peptide comprising SEQ ID NO: 4;

a third nucleic acid sequence encoding a peptide comprising SEQ ID NO: 6; and

a fourth nucleic acid sequence encoding a peptide comprising SEQ ID NO: 8

In accordance with embodiment 14, the composition of embodiment 11 is provided wherein

the first nucleic acid sequence encodes a peptide having at least 95% sequence identity to SEQ ID NO: 2;

the second nucleic acid sequence encodes a peptide having at least 95% sequence identity to SEQ ID NO: 4;

the third nucleic acid sequence encodes a peptide having at least 95% sequence identity to SEQ ID NO: 6; and said optional

a fourth nucleic acid sequence encodes a peptide having at least 95% sequence identity to SEQ ID NO: 8.

In accordance with embodiment 15, the composition of embodiment 14 is provided wherein said composition comprises said first, second, third and fourth nucleic acid sequences.

In accordance with embodiment 16, the composition of any one of embodiments 11-15 is provided wherein two or more of said first, second, third and fourth nucleic acids are part of an expression vector wherein the expression vector comprises a single eukaryotic promoter operably linked to a multiple coding sequence that comprises said two or more first, second, third and fourth nucleic acid sequences wherein said multiple coding sequence further comprises internal ribosome entry sites present before each of said two or more first, second, third and fourth nucleic acid sequences.

In accordance with embodiment 17, the composition of embodiment 16 is provided wherein said multiple coding sequence comprises all four of said first, second, third and optionally fourth nucleic acid sequence, each proceeded by an internal ribosome entry sites and operably linked to said single eukaryotic promoter.

In accordance with embodiment 18, the composition of any one of embodiments 11-17 is provided wherein the first, second, third and fourth nucleic acid sequences are part of a non-viral vector.

In accordance with embodiment 19, a kit for conducting in vivo transfection of post-natal skin tissue and inducing the skin tissue to be insulinogenic is provided, wherein said kit comprises

a disposable nanotransfection device; and

a reprogramming cocktail, wherein the reprogramming cocktail solution comprises a first nucleic acid sequence encoding for Pancreatic And Duodenal Homeobox 1 (PDX-1), a second nucleic acid sequence encoding for transcription factor MafA, a third nucleic acid sequence encoding for glucagon-like peptide 1 receptor (GLP-1R); and a fourth nucleic acid sequence comprising nucleic acid sequence encoding for Fibroblast growth factor 21 (FGF21).

In accordance with embodiment 20 the kit of embodiment 19 is provided wherein the nanotransfection device comprises a hollow microneedle array with one or more compartments for receiving said reprogramming cocktail solution.

### Example 1

### Reprogramming of Skin Tissue to be Insulinogenic

### Details of Procedure:

### In Vivo Tissue Reprogramming (Lentivirus-Mediated and TNT-Mediated)

Diabetes was induced in eight-week-old male mice (C57Bl/6, Jackson laboratories, Cat. #000664) by administering 50 mg/kg Streptozotocin (STZ, Cat. #-S0130 Millipore Sigma) via intraperitoneal injection for 5 consecutive days. The drug STZ, selectively destroys the beta cells of the pancreatic islets that leads to elevation in blood glucose (up to 400-500 mg/dL) developing diabetes in the mice. For blood glucose monitoring, mice were fasted for 06 hrs. and blood glucose was measured every 7 days using Contour blood glucose meter (Cat. #-9545C) and test strips (Cat. #-7099).

For lentivirus-mediated PMGF reprogramming, the mice in PMGF group were injected intradermally with PDX-1, MafA, GLP-1R, FGF21 overexpressing lentivirus for three days alternately (day 1, 3 and 5) at posterior dorsal skin (100 μl per mice at a titer of 10⁷ particles/mL for each reprogramming factor). The control mice were injected with control vector containing lentivirus without any reprogramming factors (100 μl per mice at a titer of 10⁷ particles/mL). Mouse lentiviruses were purchased from Applied Biological Materials Inc., Richmond, BC, Canada with Cat. #LV002.

For TNT-mediated PMGF reprogramming, the areas to be treated were first naired 24-48 h prior to TNT. The skin was then exfoliated to eliminate the dead/keratin cell layer and expose nucleated cells in the epidermis. The TNT devices were placed directly over the exfoliated skin surface. PMGF plasmid cocktails were loaded in the reservoir at a concentration of 0.05-0.1 μg/pl. A gold-coated electrode (i.e., cathode) was immersed in the plasmid solution, while a 24G needle counter-electrode (i.e., anode) was inserted intradermally, juxtaposed to the TNT platform surface. A pulsed electrical stimulation (i.e., 10 pulses of 250 V in amplitude and a duration of 10 ms per pulse) was then applied across the electrodes to nanoporate the exposed cell membranes and drive the plasmid cargo into the cells through the nanochannels. PMGF (PM:G:F) plasmids were mixed at a 1:1:1 molar ratio. In the reprogramming cocktail 37.5 μg of each component PM/G/F was used.

An Equal amount of control plasmids were delivered to Control mice group. Unless otherwise specified, control specimens involved TNT treatments with a blank, phosphate buffer saline (PBS)/mock plasmid solution. Mock (empty vector), PDX-1-MafA, GLP-1R and FGF-21 plasmids were prepared using a plasmid DNA purification kit (ZymoPURE II Plasmid Midiprep Kit, cat. no. D4201) and DNA concentrations were obtained from Nanodrop 2000c Spectrophotemeter (Thermoscientific). PDX-1-MafA, GLP-1R, FGF-21 plasmids were constructed with GFP (PDX-1-MafA), td-Tomato (GLP-1R) or CFP (FGF-21) by Applied Biological Materials Inc., Richmond, BC, Canada, Cat. #C315. For blood glucose monitoring, mice were fasted for 06 hrs. and blood glucose was measured every 7 days using Contour blood glucose meter (Cat. #-9545C) and test strips (Cat. #-7099).

### Intraperitoneal Glucose Tolerance Test (IPGTT)

IPGTT is used to test the clearance of an intraperitoneally injected glucose load from the body. This test was conducted after week 07 of TNT interventions. This test detects disturbances in glucose metabolism and insulin secretion. For this experiment mice were fasted for 06 hours and the fasting blood glucose levels were determined before a solution of glucose (D-glucose, Gibco, Cat. #15023-021, 2 g/kg of body weight) was administered by intra-peritoneal (IP) injection. Subsequently, the blood glucose level was measured from tail vein at different time points (0, 15, 30, 60, 90 and 120 minutes) during the following 120 minutes.

### Immunohistochemistry and Microscopy

For histological examination, harvested skin and pancreas from the euthanized mice were embedded in paraffin and processed for immunohistochemistry with signature antibodies of insulin-producing cells, insulin (Abcam, ab7842, 1:100 dilution) and C-peptide (Abcam, ab14181, 1:100 dilution). The signal was visualized by subsequent incubation with appropriate fluorescence-tagged secondary antibodies (Alexa 488-tagged α-guinea pig, 1:200; Alexa 568-tagged α-rabbit, 1:200) and counter-stained with DAPI. Images were captured by a laser scanning confocal microscope (Olympus FV 1000 filter/spectral).

Confocal images showed formation of insulin and C-peptide in reprogrammed skin. For histological examination, harvested skin and pancreas from the euthanized mice were embedded in paraffin and processed for immunohistochemistry with signature antibodies of insulin-producing cells, insulin (Abcam, ab7842, 1:100 dilution) and C-peptide (Abcam, ab14181, 1:100 dilution). The reprogrammed skin showed insulinogenic cells which were islet-like clusters in morphology with the production of abundant insulin and C-peptide, the signature markers of pancreatic islets beta cells. C-peptide expression in skin provides evidence of the de novo formation of insulin in reprogrammed skin. Interestingly, no such structures were found in control skin. Thus, the data indicated that the PMGF reprogramming factor cocktail leads to tissue reprogramming resulting in formation of insulinogenic cells in post-natal skin which leads to the control of blood glucose levels in streptozotocin-induced diabetic models in mice. Confocal microscopy image of mouse skin 24 hrs. after TNT treatment revealed expression of PDX-1-MafA pancreatic transcription factor.

1. A method of reprograming cells of a somatic tissue to produce insulin and C-peptide, said method comprising the step of:
delivering intracellularly into said cells of the somatic tissue DNA comprising
a first nucleic acid sequence encoding a peptide having at least 95% sequence identity to SEQ ID NO: 2;
a second nucleic acid sequence encoding a peptide having at least 95% sequence identity to SEQ ID NO: 4;
a third nucleic acid sequence encoding a peptide having at least 95% sequence identity to SEQ ID NO: 6; and optionally
a fourth nucleic acid sequence encoding a peptide having at least 95% sequence identity to SEQ ID NO: 8.

2. The method of claim 1 wherein said first, second, third and fourth nucleic acid sequences are each delivered simultaneously into the cytosol of cells of said somatic tissue in vivo.

3. The method of claim 1 or 2 wherein one or more expression vectors are transfected into said cells of the somatic tissue wherein said expression vectors comprise said first, second, third and fourth nucleic acid sequences.

4. The method of claim 2 wherein two or more of said first, second, third and fourth nucleic acids are part of an expression vector wherein the expression vector comprises a single eukaryotic promoter operably linked to a multiple coding sequence that comprises said two or more first, second, third and fourth nucleic acid sequences wherein said multiple coding sequence further comprises internal ribosome entry sites present before each of said two or more first, second, third and fourth nucleic acid sequences.

5. The method of claim 4 wherein each of said first, second, third and fourth nucleic acids are located on a single expression vector.

6. The method of claim 5 wherein the somatic cell is a skin cell.

7. The method of claim 1 wherein the intracellular delivery is via tissue nanotransfection.

8. The method of claim 7 wherein the cells are skin cells of skin tissue transfected in vivo.

9. A method of normalizing blood glucose levels in a subject with diabetes, said method comprising the step of reprogramming targeted skin tissue in vivo to produce insulin, said method comprising
contacting the cells of said target skin tissue with a reprogramming composition under conditions that enhance cellular uptake of the reprogramming composition components, wherein the reprogramming composition comprises
a first nucleic acid sequence encoding a peptide having at least 95% sequence identity to SEQ ID NO: 2;
a second nucleic acid sequence encoding a peptide having at least 95% sequence identity to SEQ ID NO: 4; and
a third nucleic acid sequence encoding a peptide having at least 95% sequence identity to SEQ ID NO: 6; and optionally
a fourth nucleic acid sequence encoding a peptide having at least 95% sequence identity to SEQ ID NO: 8 to said target skin cells.

10. A composition comprising
a first nucleic acid sequence encoding for Pancreatic And Duodenal Homeobox 1 (PDX-1);
a second nucleic acid sequence encoding for transcription factor MafA;
a third nucleic acid sequence encoding for glucagon-like peptide 1 receptor (GLP-1R); and optionally
a fourth nucleic acid sequence comprising nucleic acid sequence encoding for Fibroblast growth factor 21 (FGF21), wherein each of said first, second, third and optional fourth nucleic acid sequences are operably linked to eukaryotic regulatory sequences.

11. The composition of claim 10 wherein
the first nucleic acid sequence encodes a peptide having at least 95% sequence identity to SEQ ID NO: 2;
the second nucleic acid sequence encodes a peptide having at least 95% sequence identity to SEQ ID NO: 4;
the third nucleic acid sequence encodes a peptide having at least 95% sequence identity to SEQ ID NO: 6; and said optional
a fourth nucleic acid sequence encodes a peptide having at least 95% sequence identity to SEQ ID NO: 8.

12. The composition of claim 11 wherein said composition comprises said first, second, third and fourth nucleic acid sequences.

13. The composition of claim 12 wherein two or more of said first, second, third and fourth nucleic acids are part of an expression vector wherein the expression vector comprises a single eukaryotic promoter operably linked to a multiple coding sequence that comprises said two or more first, second, third and fourth nucleic acid sequences wherein said multiple coding sequence further comprises internal ribosome entry sites present before each of said two or more first, second, third and fourth nucleic acid sequences.

14. The composition of claim 13 wherein said multiple coding sequence comprises all four of said first, second, third and optionally fourth nucleic acid sequence, each proceeded by an internal ribosome entry sites and operably linked to said single eukaryotic promoter.

15. The composition of claim 13 or 11 wherein the first, second, third and fourth nucleic acid sequences are part of a non-viral vector.

16. A kit for conducting in vivo transfection of post-natal skin tissue and inducing the skin tissue to be insulinogenic, said kit comprising
a disposable nanotransfection device; and
a reprogramming cocktail comprising the composition of claim 10.

17. The kit of claim 16 wherein the nanotransfection device comprises a hollow microneedle array with one or more compartments for receiving said reprogramming cocktail solution.
